(19) United States
(12) Patent Application Publication
(10) Pub. No.: US 2007/0065861 A1
(43) Pub. Date: Mar. 22, 2007

(54) Methods for genetic analysis of alternative splicing

(76) Inventors: Ihor Lemischka, Princeton, NJ (US); Moshe Pritsker, Boston, MA (US)

Correspondence Address: Angela Foster, Ph.D., Esq., 2906 Birchwood Court, North Brunswick, NJ, 08902-3933, US

(21) Appl. No.: 11/524,042

(22) Filed: Sep. 19, 2006

Related U.S. Application Data
(60) Provisional application No. 60/718,353, filed on Sep. 19, 2005.

(51) Int. Cl. C12Q 1/68; C40B 30/06; C40B 40/08

(52) U.S. Cl. 435/6

(57) ABSTRACT

The present invention provides methods for large-scale functional analysis of splicing variants. Specifically, the present methods provide polynucleotide probes that incorporate exon junction sequences arising from alternative splicing events. In one embodiment of this invention, the function of a splice variant is determined by identifying a polynucleotide comprising an exon junction that binds one or more polynucleotide isolated from sample cells yet binds a reduced amount or greater amount of polynucleotide isolated from sample cells exposed to altered conditions.

### 1. RELATED APPLICATIONS

This application claims the benefit of U.S. Provisional Application No. 60/718,353 filed on Sep. 19, 2005.

### 2. FIELD OF THE INVENTION

The present invention relates to methods for identifying biological function of alternative splicing variants by using genetic screens and a variety of polynucleotide probes that span exons and potential exon-exon junctions arising from exon exclusion.

### 3. BACKGROUND OF THE INVENTION

The architecture of most genes in higher eukaryotes consists of interspersed coding exons and non-coding introns. (Sharp, 1994 Cell 77:805-814). The removal of introns and joining of exons by RNA splicing is an essential step in the assembly of functional mRNAs. Alternative splicing processes can assemble different combinations of exons to produce mRNA isoforms with distinct protein coding potentials. Recent computational studies have utilized cDNA and EST sequences to study alternative splicing on a genome-wide. (Black, 2000 Cell 103:367-370). These analyses have suggested that alternatively spliced transcripts are produced from more than 50% of human genes. (Modrek et al., 2001 Nucleic Acids. Res. 29:2850-2859). Most alternatively spliced variants occur within protein-coding regions. (Zavolan et al., 2002 Genome Res. 12:1377-1385). Alternative splicing of pre-mRNA has been proposed as a major source of genomic coding complexity. (Maniatis and Tasic, 2002 Nature 418:2386-243).

A single microarray experiment can identify thousands of genes expressed in a particular tissue or developmental stage. Several research groups have attempted to develop genomic tools for large scale detection of alternative splicing events. (Clark et al., 2002 Science 296:907-910; Johnson et al., 2003 Science 302:2141-2144; Yeakley et al., 2002 Nat. Biotechnol. 20:353-358). Yet, there is no method that provides the ability to determine function of splicing variants at the genome-wide level.

Accordingly, the present invention provides a method for identifying splicing variants responsible for a biological phenotype in a specific tissue or cell type.

Discussion or citation of a reference herein shall not be construed as an admission that such reference is prior art to the present invention.

### 4. SUMMARY OF THE INVENTION

The present inventors observed that the frequency of alternative splicing is especially high in tissue-specific genes as compared to ubiquitous genes. They also observed that alternative splicing can modify multiple components of signaling pathways important for stem function. Accordingly, the present inventors developed a method for analyzing the role of alternative splicing events. Specifically, the present invention provides microarrays that include probes complimentary to exon and exon junctions wherein the microarrays are used for parallel functional analysis of splice variants.

The survival of the cells may depend on the presence or absence of a functional cDNA or nucleic acid. The present method allows the rapid identification of splice variants that rescue cell growth at altered cell conditions. Therefore, the present invention provides a method for identifying a polynucleotide comprising an exon junction by introducing an exogeneous polynucleotide sequence into cells at starting and/or altered conditions.

Polynucleotides are isolated from these cells and probed with a polynucleotide comprising a junction resulting from exon exclusion. The function of splice variant is determined by identifying whether the exon junction binds one or more polynucleotides isolated from the starting cells and binds a reduced amount or no polynucleotides isolated from the altered cells.

In one embodiment of the present invention, the exogenous polynucleotide contains a cDNA, a fragment of a cDNA or an inhibitory polynucleotide. In another embodiment, at least one of the genetic elements includes an inhibitory polynucleotide The inhibitory polynucleotide includes an RNAi, a siRNA, a microRNA, a ribozyme RNA, an aptamer, or a DNA transcribable into any one of the RNA polynucleotides.

The cell samples may be from the same species or different species. The samples may possess different phenotypes. Accordingly, the current invention also provides a method for differentiating between one cell type and a second cell type based on tissue-dependent and organ-dependent splicing variations. A polynucleotide is identified by determining whether a probe comprising an exon junction binds one or more polynucleotides isolated from one organ and/or tissue and binds no, or a substantially reduced amount of one or more polynucleotides isolated from a different organ and/or tissue.

### 5. DETAILED DESCRIPTION OF THE FIGURES

FIG. 1 provides a schematic representation of exon-exon junctions.

FIG. 2 provides a schematic representation of a computational and experimental approach to identifying alternative splicing. The grayscale image was prepared by computer from a color original.

FIG. 3 demonstrates the correlation between transcription and frequency of alternative splicing. In Panels B and C, the grayscale images were prepared by computer from a color original. In Pane A, the exon exclusion fraction (the number of isoforms with excluded exons divided by the number of all the transcripts containing the site) is plotted versus the number of ESTs observed for a given gene. Note that genes with high numbers of ESTs (ubiquitously expressed genes over-represented in EST libraries) show a low level of exon exclusion. Representative examples of ubiquitously expressed and tissue-specific genes are shown in Panels B and C, respectively. In Panels B and C, the schematic representations of exons to the right of the gels show sequential exons with the number of bases written above each exon and possible splicing events yielding exon inclusion (upper line segments) and exon exclusion (lower line segments).

FIG. 4 demonstrates characterization of the frequency of exon exclusion. In Panels A and B, the grayscale images were prepared by computer from a color original. (Panel A) represents distribution of alternative splice sites as a function of the number of isoforms with excluded exons divided by the number of all the transcripts containing the site, i.e., the exon exclusion fraction. (panel B) represents RT-PCR profiles of alternative splicing in representative genes. The gene names follow SwissProt nomenclature. The diagrams show patterns of alternative splicing. All splicing variants have been confirmed by DNA sequencing. (Panel C) represents correspondence between experimental and computational results.

FIG. 5 demonstrates detection of alternative splicing with microarrays. The grayscale images were prepared by computer from a color original. (Panel A) represents two types of probes were designed, “exon” and “junction” probes. The exon probes are complementary to sequences of gene exons. The junction probes are complimentary to junction that potentially can be created by alternative splicing. Both types of probes are represented on microarrays. (Panel B) represents differentiation of embryonic stem (ES) cells. (Panel C) represents hybridization signal for probes for exons 1, 2, 3, and 4 for Nanog, a molecular marker of the ES cells. Line segments in the schematic exon diagram show inclusive splicing only. (Panel D) provides examples of alternative splicing detected with the novel microarrays. Line segments in the schematic diagram show both inclusive (above) and exclusive (below) splicing.

FIG. 6 provides a schematic representation of a method of detecting in differentially expressed genes. The grayscale image was prepared by computer from a color original. After transfection, vector DNA is isolated from cells, before and after imposing a differential condition. The nucleic acids collected from the cells in each sample are amplified, labeled with Cy-5 and Cy-3 and hybridized to microarrays. Changes in the level before and after the differential condition are recorded for each gene.

FIG. 7 demonstrates a stable episomal vector for cDNA over-expression analysis. The grayscale image was prepared by computer from a color original. Vector pMGD20neo constitutively expresses a non-transforming variant of polyoma virus T-protein. This protein maintains propagation of a plasmid vector, CAG-IP that carries the polyoma virus origin of replication. In addition, the CAG-IP plasmid includes a strong synthetic promoter that allows especially high expression of a downstream gene. The lower panel shows expression of the EGFP (enhanced green fluorescent protein) using this system.

### 6. DETAILED DESCRIPTION OF THE INVENTION

This section presents a detailed description of the invention and its applications. This description is by way of several exemplary illustrations, in increasing detail and specificity, of the general methods of this invention. These examples are non-limiting and related variants will be apparent to one of skill in the art.

As used herein, the term “inclusive splicing”, “exon inclusion”, and similar terms and phrases relate to a nucleic acid sequence arising from the inclusion of every exon ascribed to a given gene as the exons appear in genomic DNA. Thus, a sequence arising from inclusive splicing may be exemplified by an mRNA or its resulting cDNA that includes all exons that constitute a particular gene, as understood by one of skill in the field. The term “inclusive splicing” and similar terms and phrases, also relates to the gene product encoded by a mRNA or a cDNA resulting from inclusive splicing. Such a gene product is a polypeptide that includes amino acid sequences encoded by every exon present in genomic DNA understood by one of skill in the field, to constitute the gene for the particular polypeptide. As used herein, terms such as “contiguous splicing” and “contiguous exons”, and similar terms and phrases, are used to describe polynucleotides and their encoded polypeptides arising from inclusive splicing.

As used herein, the terms “excluded splicing”, “exon exclusion”, and similar terms and phrases, relate to a nucleic acid sequence arising from the omission of at least one exon ascribed to a given gene as those exons appear in genomic DNA, as these are understood by one of skill in the field. Thus, a sequence arising from excluded splicing may be exemplified by a mRNA or its resulting cDNA that omits at least one exon understood by one of skill in the field, to constitute a particular gene. The term “excluded splicing” and similar terms and phrases also relate to the gene product encoded by a mRNA or a cDNA resulting from excluded splicing. Such a gene product is a polypeptide that contains one or more gaps in sequence corresponding to the one or more excluded exons of the genomic DNA understood to constitute the gene for the particular polypeptide. As used herein, terms such as “gapped splicing”, “alternative splicing” and “gapped exons”, and similar terms and phrases, also are used to describe polynucleotides and their encoded polypeptides arising from excluded splicing. In addition, alternative splicing generates exon junctions resulting from the excluded exon or exons. Such junctions, termed “excluded junctions”, “junction resulting from exon exclusion”, or similar terms herein, provide nucleotide and amino acid sequences not found in the corresponding inclusively spliced nucleic acid and its gene product.

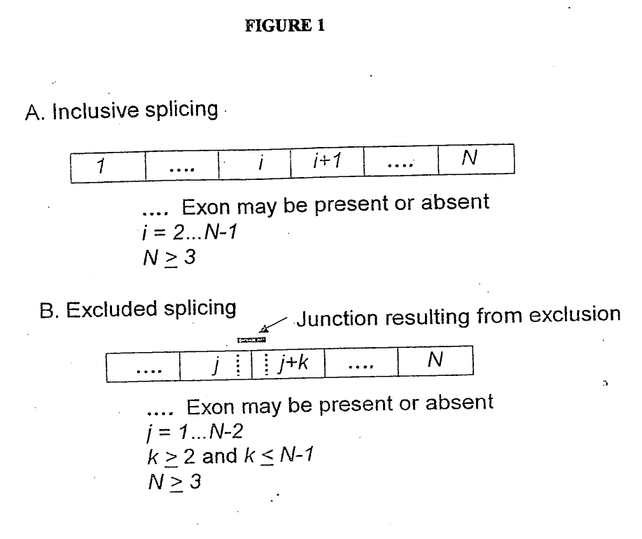

FIG. 1 provides a schematic representation of inclusive splicing versus excluded splicing. In Panel A, inclusive splicing is shown schematically for a gene having N exons, where N is greater than 2. The index i is used to enumerate the exons between the first and the last exon. Panel B schematically illustrates a case of excluded splicing for a gene having N exons, where N is greater than 2. The index j is used to enumerate exons and the index k is used to indicate the next exon appearing after the j-th exon resulting from the exclusion of one or more exons. The horizontal bar over the junction between the j-th and j+k-th exon, as well as the vertical dotted lines positioned within the exon below the ends of the junction bar, characterizes a probe that could be used to identify a polynucleotide resulting from exon exclusion at that site.

As used herein, the term “mono-exonic sequence”, “intra-exonic sequence”, and similar terms and phrases relates to a polynucleotide and its encoded polypeptide that includes a sequence contained entirely within the bounds of a single known exon.

As used herein, the term “inhibitory” polynucleotide and similar terms and phrases relate to a polynucleotide sequence that is effective to inhibit the transcriptional or translational expression of a target polynucleotide. Non-limiting examples of inhibitory polynucleotides include antisense nucleic acids, short inhibitory RNAs (siRNAs), microRNAs, ribozymes, aptamers, and so forth. Any equivalent inhibitory polynucleotide is encompassed within the scope of the present invention.

As used herein, the term “homologous sequence” and similar terms and phrases relate to all the known or possible members of a family of nucleic acids that includes the sequence arising from inclusive splicing as well as from any and all alternative splicing, or excluded splicing, events with respect to the genomic DNA of a particular species of organism. A homologous sequence as used herein, also applies to a gene product encoded by any member of a family of homologous nucleic acids.

As used herein the term “present” and similar terms and phrases, when applied to a nucleic acid, a polynucleotide, and oligonucleotide, a protein, a polypeptide, or an oligopeptide, relates to a finding that the substance in question is detectable to an extent at least two-fold greater than a limit of detection for the substance when using a particular method of detection. As used herein, the term “substantially absent” and similar terms and phrases, when applied to a nucleic acid, a polynucleotide, and oligonucleotide, a protein, a polypeptide, or an oligopeptide, relates to a finding that the substance in question is undetectable or barely detectable at the limit of detection for the substance when using a particular method of detection.

6.1 Polynucleotides

As used herein, the terms “nucleic acid” and “polynucleotide” and similar terms and phrases are considered synonymous with each other, and are used as conventionally understood by workers of skill in fields such as biochemistry, molecular biology, genomics, and similar fields related to the field of the invention. A polynucleotide employed in the invention may be single stranded or it may be a base paired double stranded structure, or even a triple stranded base paired structure. A polynucleotide may be a DNA, RNA, or any mixture or combination of a DNA strand and RNA strand, such as, by way of non-limiting example, a DNA-RNA duplex structure. A polynucleotide and an “oligonucleotide” as used herein are identical in any and all attributes defined here for a polynucleotide except for the length of a strand. As used herein, a polynucleotide may be about 50 nucleotides or base pairs in length or longer, or may be of the length of, or longer than, about 60, or about 70, or about 80, or about 100, or about 150, or about 200, or about 300, or about 400, or about 500, or about 700, or about 1000, or about 1500, or about 2000 or about 2500, or about 3000, nucleotides or base pairs or even longer. An oligonucleotide may be at least 3 nucleotides or base pairs in length, and may be shorter than about 70, or about 60, or about 50, or about 40, or about 30, or about 20, or about 15, or about 10 nucleotides or base pairs in length. Both polynucleotides and oligonucleotides may be chemically synthesized. Oligonucleotides may be used as probes. As used herein, a polynucleotide, an oligonucleotide or a probe nucleic acid may arise from inclusive splicing events or from excluded splicing events.

As used herein “fragment” and similar words relate to portions of a nucleic acid, polynucleotide or oligonucleotide, or to portions of a protein or polypeptide, shorter than the full sequence of a reference. The sequence of bases or the sequence of amino acid residues, in a fragment is unaltered from the sequence of the corresponding portion of the molecule from which it arose. There are no insertions or deletions in a fragment in comparison with the corresponding portion of the molecule from which it arose. As contemplated herein, a fragment of a nucleic acid or polynucleotide, such as an oligonucleotide, is 15 or more bases in length, or 16 or more, 17 or more, 18 or more, 21 or more, 24 or more, 27 or more, 30 or more, 50 or more, 75 or more, 100 or more bases in length, up to a length that is one base shorter than the full length sequence. Any fragment of a polynucleotide may be chemically synthesized and may be used as a probe.

As used herein and in the claims “nucleotide sequence”, “oligonucleotide sequence” or “polynucleotide sequence”, “polypeptide sequence”, “amino acid sequence”, “peptide sequence”, “oligopeptide sequence”, and similar terms, relate interchangeably both to the sequence of bases or amino acids that an oligonucleotide or polynucleotide, or polypeptide, peptide or oligopeptide has, as well as to the oligonucleotide or polynucleotide, or polypeptide, peptide or oligopeptide structure possessing the sequence. A nucleotide sequence or a polynucleotide sequence, or polypeptide sequence, peptide sequence or oligopeptide sequence furthermore relates to any natural or synthetic polynucleotide or oligonucleotide, or polypeptide, peptide or oligopeptide, in which the sequence of bases or amino acids is defined by description or recitation of a particular sequence of letters designating bases or amino acids as conventionally employed in the field.

Nucleotide residues occupy sequential positions in an oligonucleotide or a polynucleotide. Accordingly, a modification or derivative of a nucleotide may occur at any sequential position in an oligonucleotide or a polynucleotide. All modified or derivatized oligonucleotides and polynucleotides are encompassed within the invention and fall within the scope of the claims. Modifications or derivatives can occur in the phosphate group, the monosaccharide or the base. Such modifications include, by way of non-limiting example, modified bases and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

As used herein and in the claims, a “nucleic acid” or “polynucleotide”, and similar terms based on these, refer to polymers composed of naturally occurring nucleotides as well as to polymers composed of synthetic or modified nucleotides. Thus, as used herein, a polynucleotide that is a RNA or DNA, may include naturally occurring moieties such as the naturally occurring bases and ribose or deoxyribose rings, or they may be composed of synthetic or modified moieties as described in the following. The linkage between nucleotides is commonly the 3′-5′ phosphate linkage, which may be a natural phosphodiester linkage, a phosphothioester linkage, and other synthetic linkages. Examples of modified backbones include, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates. Additional linkages include phosphotriester, siloxane, carbonate, carboxymethylester, acetamidate, carbamate, thioether, bridged phosphoramidate, bridged methylene phosphonate, bridged phosphorothioate and sulfone internucleotide linkages. Other polymeric linkages include 2′-5′ linked analogs of these. (see U.S. Pat. Nos. 6,503,754 and 6,506,735). The monosaccharide may be modified by being, for example, a pentose or a hexose other than a ribose or a deoxyribose. The monosaccharide may also be modified by substituting hydryoxyl groups with hydro or amino groups, by esterifying additional hydroxyl groups, and so on.

The bases in oligonucleotides and polynucleotides may be “unmodified” or “natural” bases include the purine bases adenine (A) and guanine (G), and the pyrimidine bases thymine (T), cytosine (C) and uracil (U). In addition, they may be bases with modifications or substitutions. As used herein, modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-fluoro-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1-pyrimido[5,4-b][1,4]benzothiazin-2(3H)-one), G-clamps such as a substituted phenoxazine cytidine (e.g., 9-(2-aminoethoxy)-H-pyrimido[5,4-b][1,4]benzoxazin-2(3H)-one), carbazole cytidine (2H-pyrimido[4,5-b]indol-2-one), pyridoindole cytidine (H-pyrido[3′, 2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808; The Concise Encyclopedia Of Polymer Science And Engineering, pages 858-859, Kroschwitz, J. I., ed. John Wiley & Sons, 1990; Englisch et al., Angewandte Chemie, International Edition (1991) 30, 613; and Sanghvi, Y. S., Chapter 15, Antisense Research and Applications, pages 289-302, Crooke, S. T. and Lebleu, B., ed., CRC Press, 1993. Certain of these bases are particularly useful for increasing the binding affinity of the oligomeric compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (see Sanghvi, Y. S., Crooke, S. T. and Lebleu, B., eds., Antisense Research and Applications, CRC Press, Boca Raton, 1993, pp. 276-278) and are presently preferred base substitutions, even more particularly when combined with 2′-O-methoxyethyl sugar modifications. (see U.S. Pat. Nos. 6,503,754 and 6,506,735).

Nucleotides may also be modified to harbor a label. Nucleotides bearing a fluorescent label or a biotin label, for example, are available from Sigma (St. Louis, Mo.).

As used herein an “isolated” nucleic acid molecule is one that is separated from at least one other nucleic acid molecule that is present in the natural source of the nucleic acid. Examples of isolated nucleic acid molecules include, but are not limited to, recombinant polynucleotide molecules, recombinant polynucleotide sequences contained in a vector, recombinant polynucleotide molecules maintained in a heterologous host cell, partially or substantially purified nucleic acid molecules, and synthetic DNA or RNA molecules. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 50 kb, 25 kb, 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the present invention, e.g., a nucleic acid molecule having a given nucleotide sequence, or a complement of this nucleotide sequence, can be isolated using standard molecular biology techniques and the sequence information provided herein. Using all or a portion of the nucleic acid sequence of any polynucleotide as a hybridization probe, nucleic acid sequences can be isolated using standard hybridization and cloning techniques (e.g., as described in Sambrook et al., eds., Molecular Cloning: A Laboratory Manual 3rd Ed., Cold Spring Harbor Laboratory Press, Cold Spring Harbor, N.Y., 2001; and Brent et al., Current Protocols in Molecular Biology, Wiley Interscience Publishers, (2003)).

A polynucleotide or oligonucleotide, including a polynucleotide or oligonucleotide probe, may be synthesized in accordance with well-known chemical processes, including, but not limited to sequential addition of nucleotide phosphoramidites to particle-bound hydroxyl groups, as described by T. Brown and Dorcas J. S. Brown in Oligonucleotides and Analogues A Practical Approach, F. Eckstein, editor, Oxford University Press, Oxford, pp. 1-24 (1991), and incorporated herein by reference. Other methods of oligonucleotide synthesis include, but are not limited to solid-phase oligonucleotide synthesis according to the phosphotriester and phosphodiester methods (Narang, et al., (1979) Meth. Enzymol. 68:90), and to the H-phosphonate method (Garegg, P. J., et al., (1985) “Formation of internucleotidic bonds via phosphonate intermediates”, Chem. Scripta 25, 280-282; and Froehler, B. C., et al., (1986a) “Synthesis of DNA via deoxynucleoside H-phosphonate intermediates”, Nucleic Acid Res., 14, 5399-5407, among others) and synthesis on a support (Beaucage, et al. (1981) Tetrahedron Letters 22:1859-1862) as well as phosphoramidate techniques (Caruthers, M. H., et al., (1988) Methods in Enzymology, Vol. 154, pp. 287-314), U.S. Pat. Nos. 5,153,319; 5,132,418; 4,500,707; 4,458,066; 4,973,679; 4,668,777; and 4,415,732, and others described in “Synthesis and Applications of DNA and RNA,” S. A. Narang, editor, Academic Press, New York, 1987, and the references contained therein, and nonphosphoramidite techniques.

As used herein, the term “complementary” refers to Watson-Crick or Hoogsteen base pairing between nucleotides units of a nucleic acid molecule. As used herein and in the claims, the term “complementary” and similar words, relate to the ability of a first nucleic acid base in one strand of a nucleic acid, polynucleotide or oligonucleotide to interact specifically only with a particular second nucleic acid base in a second strand of a nucleic acid, polynucleotide or oligonucleotide. By way of non-limiting example, if the naturally occurring bases are considered, A and T or U interact with each other, and G and C interact with each other. As employed in this invention and in the claims, “complementary” is intended to signify “fully complementary” within a region, namely, that when two polynucleotide strands are aligned with each other, at least in the region each base in a sequence of contiguous bases in one strand is complementary to an interacting base in a sequence of contiguous bases of the same length on the opposing strand.

As used herein, “hybridize”, “hybridization” and similar words relate to a process of forming a nucleic acid, polynucleotide, or oligonucleotide duplex by causing strands with complementary sequences to interact with each other. The interaction occurs by virtue of complementary bases on each of the strands specifically interacting to form a pair. The ability of strands to hybridize to each other depends on a variety of conditions, as set forth below. Nucleic acid strands hybridize with each other when a sufficient number of corresponding positions in each strand are occupied by nucleotides that can interact with each other. It is understood by workers of skill in the field of the present invention, including by way of non-limiting example molecular biologists and cell biologists, that the sequences of strands forming a duplex need not be 100% complementary to each other to be specifically hybridizable.

In another embodiment, an isolated nucleic acid molecule of the invention comprises a nucleic acid molecule that is a complement of a given nucleotide sequence, or a portion of this nucleotide sequence. A nucleic acid molecule that is complementary to a given nucleotide sequence is one that is sufficiently complementary to the given nucleotide sequence that it can hydrogen bond with few or no mismatches to the given nucleotide sequence, thereby forming a stable duplex.

A significant use of a nucleic acid, polynucleotide, or oligonucleotide is in an assay directed to identifying a target sequence to which a probe nucleic acid hybridizes. The selectivity of a probe for a target is affected by the stringency of the hybridizing conditions. “Stringency” of hybridization reactions is readily determinable by one of ordinary skill in the art, and generally is an empirical evaluation dependent upon probe length, temperature, and buffer composition. Hybridization generally depends on the ability of denatured DNA to re-anneal when complementary strands are present in an environment below their melting temperature. Higher relative temperatures tend to make the reaction conditions more stringent, while lower temperatures less so. For additional details and explanation of stringency of hybridization reactions and identifying hybridization conditions of varying stringency, see Brent et al., Current Protocols in Molecular Biology, Wiley Interscience Publishers, (2003), and Sambrook et al., Molecular Cloning: A Laboratory Manual, 3^{rd} Ed., New York: Cold Spring Harbor Press, 2001. In addition, in high throughput or multiplexed assay systems, both the probe characteristics and the stringency may be optimized to permit achieving the objectives of the multiplexed assay under a single set of stringency conditions.

Non-limiting examples of “stringent conditions” or “high stringency conditions”, as defined herein, include those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; (3) employ 50% formamide, 5×SSC (0.75 M NaCl, 0.075 M sodium citrate), 50 mM sodium phosphate (pH 6.8), 0.1% sodium pyrophosphate, 5× Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C., or (4) employ 7% sodium dodecyl sulfate (SDS), 0.5 M NaPO₄, 1 mM EDTA at 50° C. with washing in 2×SSC, 0.1% SDS at 50° C.

“Moderately stringent conditions” include, by way of non-limiting example, the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5× Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

6.2 Polynucleotide Libraries

As used herein, a “polynucleotide library” and similar terms and phrases relates to a population of polynucleotides the members of which include nucleotide sequences that differ from one another. In many embodiments, the members of a library contain coding sequences that differ from one another, or fragments thereof that differ from one another. An important example of a library as used herein is a cDNA library. Such a library is prepared from the nucleic acids isolated from a given cell in culture, or the cells of a tissue, or the cells of an organ, such that the resulting library includes many cDNAs representing expressed genes present in the cell, tissue or organ. In many cases, cDNA libraries from desired sources are available from commercial suppliers. For many purposes useful in the present disclosure, polynucleotide libraries may be incorporated into a plasmid, to provide a library of plasmids, for transfection into a host cell. A polynucleotide library may be a library of antisense polynucleotides or a library of interfering polynucleotides.

As used herein, the terms “inhibitory polynucleotide”, “interfering polynucleotide”, and related terms and phrases, relate to any polynucleotide or any oligonucleotide that is effective to inhibit or to interfere with the expression of a coding sequence contained in a “target” polynucleotide sequence. By way of non-limiting example, an inhibitory polynucleotide may be an antisense polynucleotide, an interfering polynucleotide such as an interfering RNA or a DNA that may be transcribed into or be processed to provide an interfering RNA intracellularly, a ribozyme or a DNA providing a ribozyme RNA sequence, an aptamer, a triple helical polynucleotide, and the like. Any equivalent inhibitory polynucleotide or interfering polynucleotide is encompassed within scope of the instant disclosure.

6.3 Variant Polynucleotide

The invention further encompasses nucleic acid molecules that differ from a disclosed nucleotide sequences. For example, a sequence may differ due to degeneracy of the genetic code. These nucleic acids encode the same protein as that encoded by the disclosed nucleotide sequence. In such embodiments, an isolated nucleic acid molecule of the invention has a nucleotide sequence encoding a protein having an amino acid sequence encoded by the given or disclosed polynucleotide.

In addition to the nucleotide sequence of a given polynucleotide, it will be appreciated by those skilled in the art that DNA allelic sequence polymorphisms that lead to changes in the amino acid sequences of protein may exist within a population (e.g., the human population). Such natural allelic variations can typically result in 1-5% variance in the nucleotide sequence of the gene. Any and all such nucleotide variations and resulting amino acid polymorphisms in the protein that are the result of natural allelic variation and that do not alter the functional activity of the protein are intended to be within the scope of the invention.

Moreover, nucleic acid molecules encoding orthologs from other species and that have a nucleotide sequence that differs from a disclosed sequence are intended to be within the scope of the invention. Nucleic acid molecules corresponding to natural allelic variants and orthologs of the cDNAs of the invention can be isolated based on their homology to the human nucleic acids disclosed herein using the human cDNAs, or a portion thereof, as a hybridization probe according to standard hybridization techniques under stringent hybridization conditions.

6.4 Conservative Mutations

In addition to naturally-occurring allelic variants of the sequence that may exist in the population, the skilled artisan will further appreciate that variants of a disclosed nucleotide sequence can be generated by a skilled artisan, thereby leading to changes in the amino acid sequence of the encoded protein, without altering the functional ability of the protein. For example, nucleotide substitutions leading to amino acid substitutions at “non-essential” amino acid residues can be made in a particular disclosed sequence. A “non-essential” amino acid residue is a residue at a position in the sequence that can be altered from the wild-type sequence of the protein without altering the biological activity of the resulting gene product, whereas an “essential” amino acid residue is a residue at a position that is required for biological activity. For example, amino acid residues that are invariant among members of a family of proteins, of which the proteins of the present invention are members, are predicted to be particularly unamenable to alteration. Whether a position in an amino acid sequence of a polypeptide is invariant or subject to substitution is readily apparent upon examination of a multiple sequence alignment of homologs, orthologs and paralogs of the polypeptide.

Thus, an important aspect of the invention pertains to nucleic acid molecules encoding proteins that contain changes in amino acid residues that are not essential for activity. Such proteins differ in amino acid sequence from any given amino acid sequence yet retain biological activity. In one embodiment, the isolated nucleic acid molecule comprises a nucleotide sequence encoding a protein, wherein the protein comprises an amino acid sequence at least about 75% similar to the disclosed amino acid sequence. Preferably, the protein encoded by the nucleic acid is at least about 80% identical to a given amino acid sequence, more preferably at least about 85%, at least about 90%, at least about 95%, at least about 97%, at least about 98%, and most preferably at least about 99%/o identical to the given sequence. An isolated nucleic acid molecule encoding a protein similar to the disclosed protein can be created by introducing one or more nucleotide substitutions, additions or deletions into the corresponding nucleotide sequence, such that one or more amino acid substitutions, additions or deletions are introduced into the encoded protein.

Preferably, conservative amino acid substitutions are made at one or more predicted non-essential amino acid residues. A “conservative amino acid substitution” is one in which the amino acid residue is replaced with an amino acid residue having a similar side chain. Families of amino acid residues having similar side chains have been defined in the art. Certain amino acids have side chains with more than one classifiable characteristic, such as polar amino acid with a long aliphatic side chain. The amino acid families include amino acids with basic side chains (e.g., lysine, arginine, histidine), acidic side chains (e.g., aspartic acid, glutamic acid), uncharged polar side chains (e.g., asparagine, glutamine, serine, threonine, tyrosine, tryptophan, cysteine), nonpolar side chains (e.g., glycine, alanine, valine, leucine, isoleucine, proline, phenylalanine, methionine, tyrosine, tryptophan, lysine), beta-branched side chains (e.g., threonine, valine, isoleucine) aromatic side chains (e.g., tyrosine, phenylalanine, tryptophan, histidine) and metal-complexing side chains (e.g., aspartic acid, glutamic acid, asparagine, glutamine, serine, threonine, tyrosine, cysteine, methionine and histidine). Mutations can be introduced into a particular amino acid sequence by standard techniques, such as site-directed mutagenesis and PCR-mediated mutagenesis. Alternatively, in another embodiment, mutations can be introduced randomly along all or part of a coding sequence, such as by saturation mutagenesis, and the resultant mutants can be screened for protein biological activity to identify mutants that retain activity. Following mutagenesis the encoded protein can be expressed by any recombinant technology known in the art and the activity of the protein can be determined.

6.5 Determining Similarity Between Two or More Sequences

To determine the percent similarity of two amino acid sequences or of two nucleic acids, the sequences are aligned for optimal comparison purposes (e.g., gaps can be introduced in either of the sequences being compared for optimal alignment between the sequences). As used herein amino acid or nucleotide “identity” is synonymous with amino acid or nucleotide “homology”.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T or U, C, G, or L in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region. In polypeptides the “percentage of positive residues” is calculated by comparing two optimally aligned sequences over that region of comparison, determining the number of positions at which the identical and conservative amino acid substitutions, as defined above, occur in both sequences to yield the number of matched positions, dividing the number of matched positions by the total number of positions in the region of comparison (i.e., the window size), and multiplying the result by 100 to yield the percentage of positive residues.

“Identity,” as known in the art, is a relationship between two or more polypeptide sequences or two or more polynucleotide sequences, as determined by, comparing the sequences. In the art, “identity” also means the degree of sequence relatedness between polypeptide or polynucleotide sequences, as the case may be, as determined by the match between strings of such sequences. “Identity” and “similarity” can be readily calculated by known methods, including but not limited to those described in Computational Molecular Biology, Lesk. A. M., ed., Oxford University Press, New York, 1988; Biocomputing: Informatics and Genome Projects, Smith, D. W., ed., Academic Press, New York, 1993; Computer Analysis of Sequence Data, Part I. Griffin, A. M., and Griffin, H. G., eds. Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press. New York, 1991; and Carillo, H., and Lipman, D., SIAM J. Applied Math. (1988) 48: 1073. Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devercux, J., et al. (1984) Nucleic Acids Research 12(1): 387), BLASTP, BLASTN, and FASTA (Atschul, S. F. et al. (1990) J. Molec. Biol. 215: 403410. The BLAST X program is publicly available from NCBI and other sources (BLAST Manual, Altschul, S., et al., NCBI NLM NIH Bethesda, Md. 20894; Altschul, S., et al. (1990) J. Mol. Biol. 215: 403410. The well known Smith Waterman algorithm may also be used to determine identity.

Additionally, the BLAST alignment tool is useful for detecting similarities and percent identity between two sequences. BLAST is available on the World Wide Web at the National Center for Biotechnology Information site. References describing BLAST analysis include Madden, T. L., Tatusov, R. L. & Zhang, J. (1996) Meth. Enzymol. 266:131-141; Altschul, S. F., Madden, T. L., Schäffer, A. A., Zhang, J., Zhang, Z., Miller, W. & Lipman, D. J. (1997) Nucleic Acids Res. 25:3389-3402; and Zhang, J. & Madden, T. L. (1997) Genome Res. 7:649-656.

6.6 Antisense Nucleic Acids

Another aspect of the invention pertains to isolated antisense nucleic acid molecules that are hybridizable to or complementary to the nucleic acid molecule comprising a given nucleotide sequence, or variants, fragments, analogs or derivatives thereof. An “antisense” nucleic acid comprises a nucleotide sequence that is complementary to a “sense” nucleic acid encoding a protein, e.g., complementary to the coding strand of a double-stranded cDNA molecule or complementary to an mRNA sequence. In specific aspects, antisense nucleic acid molecules are provided that comprise a sequence complementary to a portion of at least about 10, 25, 50, 100, 250 or 500 nucleotides or an entire coding strand.

In one embodiment, an antisense nucleic acid molecule is antisense to a “coding region” of the coding strand of a nucleotide sequence encoding a protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding a protein. The term “noncoding region” refers to 5′ and 3′ sequences which flank the coding region that are not translated into amino acids (i.e., also referred to as 5′ and 3′ untranslated regions), but that may contain sequences regulating expression.

Given the coding strand sequences encoding a disclosed protein, antisense nucleic acids of the invention can be designed according to the rules of Watson and Crick or Hoogsteen base pairing. The antisense nucleic acid molecule can be complementary to the entire coding region of a mRNA, but more preferably is an oligonucleotide that is antisense to only a portion of the coding or noncoding region of a mRNA.

The antisense nucleic acid molecules of the invention are typically administered to a subject or generated in situ such that they hybridize with or bind to cellular mRNA and/or genomic DNA encoding a protein to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix.

6.7 Interfering RNA

In one aspect of the invention, gene expression can be attenuated by RNA interference. One approach well-known in the art is short interfering RNA (siRNA) or micro RNA (also designated as an interfering polynucleotide or a micro polynucleotide herein) mediated gene silencing where expression products of a gene are targeted by specific double stranded derived siRNA nucleotide sequences that are complementary to at least a 19-25 nt long segment of the gene transcript, including the 5′ untranslated (UT) region, the ORF, or the 3′ UT region. (See, e.g., PCT applications WO00/44895, WO99/32619, WO01n75164, WO01/92513, WO 01/29058, WO01/89304, WO02/16620, and WO02/29858; see also, Jia et al., (2003) J. Virol. 77(5):3301-3306, and Morris et al., (2004) Science 305:1289-1292). Targeted genes can be a gene, or an upstream or downstream modulator of the gene. Non-limiting examples of upstream or downstream modulators of a gene include, e.g., a transcription factor that binds the gene promoter, a kinase or phosphatase that interacts with a polypeptide, and polypeptides involved in a regulatory pathway.

A polynucleotide according to the invention includes a siRNA polynucleotide. Such a siRNA can be obtained using a polynucleotide sequence, for example, by processing the ribopolynucleotide sequence in a cell-free system, by transcription of recombinant double stranded RNA or by chemical synthesis of nucleotide sequences similar to a sequence. (See, e.g., Tuschl, Zamore, Lehmann, Bartel and Sharp (1999) Genes & Dev. 13: 3191-3197).

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides.

In order to generate siRNA, a contemplated recombinant expression vector of the invention comprises a DNA molecule cloned into an expression vector comprising operatively-linked regulatory sequences flanking the sequence in a manner that allows for expression of both strands. The sense and antisense RNA strands may hybridize in vivo to generate siRNA constructs for silencing of the gene by cleavage of the RNA to form siRNA molecules. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is similar to all or a portion of the target gene. In another example, a hairpin RNAi product is a siRNA. The regulatory sequences flanking the sequence may be identical or may be different, such that their expression may be modulated independently, or in a temporal or spatial manner.

In a specific embodiment, siRNAs are transcribed intracellularly by cloning the gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from Imgenex). The U6 and H1 promoters are members of the type III class of Pol III promoters.

A siRNA vector has the advantage of providing long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are digested from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A mRNA region to be targeted by siRNA is generally selected from a desired sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. (See, Elbashir et al. (2001) EMBO J. 20(23):6877-88). Hence, consideration should be taken to accommodate SNPs, polymorphisms, allelic variants or species-specific variations when targeting a desired gene.

An experiment involving a siRNA includes the proper negative control. Typically, one would scramble the nucleotide sequence of the siRNA and do a homology search to make sure it lacks homology to any other gene.

An inventive therapeutic method of the invention contemplates administering a siRNA construct as therapy to compensate for increased or aberrant expression or activity. The ribopolynucleotide is obtained and processed into siRNA fragments, or a siRNA is synthesized, as described above. The siRNA is administered to cells or tissues using known nucleic acid transfection techniques, as described above. A siRNA specific for a gene will decrease or knockdown transcription products, which will lead to reduced polypeptide production, resulting in reduced polypeptide activity in the cells or tissues.

Additional properties and uses of RNAi are reviewed in Mello, C. C. and Conte, D., Jr. (2004) Nature 431:338-342; Meister, G. and Tuschl, T. (2004) Nature 431:343-349; Ambros, V. (2004) Nature 431:350-355; Lippman, Z. and Martienssen, R. (2004) Nature 431:364-370; and Hannon, G. J., and Rossi, J. J. (2004) Nature 431:371-378.

6.8 Ribozymes

The polynucleotides contemplated herein may also be ribozymes, i.e., enzymatic RNA molecules, that may be used to inhibit gene expression by catalyzing the specific cleavage of RNA. The mechanism of ribozyme action involves sequence-specific hybridization of the ribozyme molecule to complementary target RNA, followed by endonucleolytic cleavage. Examples which may be used include engineered “hammerhead” or “hairpin” motif ribozyme molecules that can be designed to specifically and efficiently catalyze endonucleolytic cleavage of gene sequences. Ribozymes can be synthesized to recognize specific nucleotide sequences of a protein of interest and cleave it. (See Cech. J. Amer. Med Assn. 260:3030 (1988)). Techniques for the design of such molecules for use in targeted inhibition of gene expression are well known to one of skill in fields related to the present invention.

Ribozyme methods include exposing a cell to ribozymes or inducing expression in a cell of such small RNA ribozyme molecules. (See Grassi and Marini, (1996) Annals of Medicine 28:499-510; Gibson, (1996) Cancer and Metastasis Reviews 15:287-299). Intracellular expression of hammerhead and hairpin ribozymes targeted to mRNA corresponding to at least one of the genes discussed herein can be utilized to inhibit protein encoded by the gene.

Ribozymes can either be delivered directly to cells, in the form of RNA oligonucleotides incorporating ribozyme sequences, or introduced into the cell as an expression vector encoding the desired ribozymal RNA. Ribozymes can be routinely expressed in vivo in sufficient number to be catalytically effective in cleaving mRNA, and thereby modifying mRNA abundance in a cell. (see Cotten et al., (1989) EMBO J. 8:3861-3866).

6.9 Aptamers

RNA aptamers can also be introduced into or expressed in a cell to modify RNA abundance or activity. RNA aptamers are specific RNA ligands for proteins, such as for Tat and Rev RNA, that can specifically inhibit their translation. (See Good et al., (1997) Gene Therapy 4:45-54).

6.10 Triple Helical Polynucleotides

Inhibition of gene expression may be achieved using “triple helix” base-pairing methodology. Triple helix pairing is useful because it causes inhibition of the ability of the double helix to open sufficiently for the binding of polymerases, transcription factors, or regulatory molecules. Recent therapeutic advances using triplex DNA have been described in the literature. (See Gee, J. E. et al. (1994) In: Huber, B. E. and B. I. Carr, Molecular and Immunologic Approaches, Futura Publishing Co., Mt. Kisco, N.Y.). These molecules may also be designed to block translation of mRNA by preventing the transcript from binding to ribosomes.

All polynucleotides, including antisense molecules, triple helix DNA, RNA aptamers and ribozymes of the present invention may be prepared by any method known in the art for the synthesis of nucleic acid molecules. These include techniques for chemically synthesizing oligonucleotides such as solid phase phosphoramidite chemical synthesis. Alternatively, RNA molecules may be generated by in vitro and in vivo transcription of DNA sequences encoding the genes of the polypeptides discussed herein. Such DNA sequences may be incorporated into a wide variety of vectors with suitable RNA polymerase promoters such as T7 or SP6. Alternatively, cDNA constructs that synthesize antisense RNA constitutively or inducibly can be introduced into cell lines, cells, or tissues.

6.11 Production of RNAs

Sense RNA (ssRNA) and antisense RNA (asRNA) of are produced using known methods such as transcription in RNA expression vectors. See, e.g., Sambrook et al., Molecular Cloning, 3rd Ed., Cold Spring Harbor Laboratory Press, Plainview, N.Y. (2001). siRNAs, such as 21 nt RNAs, are chemically synthesized using Expedite RNA phosphoramidites and thymidine phosphoramidite Proligo, Germany). Synthetic oligonucleotides are deprotected and gel-purified (Elbashir et al. (2001) Genes & Dev. 15, 188-200), followed by Sep-Pak C18 cartridge (Waters, Milford, Mass., USA) purification (see Tuschl et al. (1993) Biochemistry, 32:11658-11668). The RNA single strands are annealed by incubating in annealing buffer (100 mM potassium acetate, 30 mM HEPES-KOH at pH 7.4, 2 mM magnesium acetate) for 1 min at 90° C. followed by 1 h at 37° C.

6.12 PNA Moieties

In various embodiments, the nucleic acids can be modified to generate peptide nucleic acids (see Hyrup et al., 1996 Bioorg Med Chem 4: 5-23). As used herein, the terms “peptide nucleic acids” or “PNAs” refer to nucleic acid mimics, e.g., DNA mimics, in which the deoxyribosephosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup et al., (1996) Bioorg Med Chem 4:5-23; Perry-O'Keefe et al., (1996) Proc. Natl. Acad. Sci. USA 93:14670-675.

PNAs can be used in therapeutic and diagnostic applications. For example, PNAs can be used as antisense or anti-gene agents for sequence-specific modulation of gene expression by, e.g., inducing transcription or translation arrest or inhibiting replication. PNAs of the proteins can also be used, e.g., in the analysis of single base pair mutations in a gene by, e.g., PNA directed PCR clamping; as artificial restriction enzymes when used in combination with other enzymes, e.g., S1 nucleases (Hyrup et al., (1996) Bioorg Med Chem 4:5-23); or as probes or primers for DNA sequence and hybridization, (Hyrup et al., (1996) Bioorg Med Chem 4:5-23; Perry-O'Keefe et al., (1996) Proc. Natl. Acad. Sci. USA 93: 14670-675).

6.13 Polypeptides

As used herein the term “protein”, “polypeptide”, or “oligopeptide”, and similar words based on these, relate to polymers of alpha amino acids joined in peptide linkage. Alpha amino acids include those encoded by triplet codons of nucleic acids, polynucleotides and oligonucleotides. They may also include amino acids with side chains that differ from those encoded by the genetic code.

As used herein, a “mature” form of a polypeptide or protein disclosed in the present invention is the product of a naturally occurring polypeptide or precursor form or proprotein. The naturally occurring polypeptide, precursor or proprotein includes, by way of non-limiting example, the full length gene product, encoded by the corresponding gene. Alternatively, it may be defined as the polypeptide, precursor or proprotein encoded by an open reading frame described herein. The product “mature” form arises, again by way of non-limiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an open reading frame, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

As used herein an “amino acid” designates any one of the naturally occurring alpha-amino acids that are found in proteins. In addition, the term “amino acid” designates any nonnaturally occurring amino acids known to workers of skill in protein chemistry, biochemistry, and other fields related to the present invention. These include, by way of nonlimiting example, sarcosine, hydroxyproline, norleucine, alloisoleucine, cyclohexylalanine, phenylglycine, homocysteine, dihydroxyphenylalanine, ornithine, citrulline, D-amino acid isomers of naturally occurring L-amino acids, and others. In addition an amino acid may be modified or derivatized, for example by coupling the side chain with a label. Any amino acid known to one of skill in the art may be incorporated into a polypeptide disclosed herein.

Peptides, oligopeptides and polypeptides may be synthesized using stepwise chain extension by well known techniques initially developed by B. Merrifield, and described, by way of nonlimiting example, in The Practice of Peptide Synthesis, 2^{nd} Ed., M Bodanszky and A. Bodanszky, Springer-Verlag, New York, N.Y. (1994).

The term “epitope tagged” when used herein refers to a chimeric polypeptide comprising a polypeptide fused to a “tag polypeptide”. The tag polypeptide has enough residues to provide an epitope against which an antibody can be made, yet is short enough such that it does not interfere with activity of the polypeptide to which it is fused. The tag polypeptide preferably also is fairly unique so that the antibody does not substantially cross-react with other epitopes. Suitable tag polypeptides generally have at least six amino acid residues and usually between about 8 and 50 amino acid residues (preferably, between about 10 and 20 amino acid residues). As used herein, the terms “active” or “activity” and similar terms refer to form(s) of a polypeptide which retain a biological and/or an immunological activity of a given native or naturally-occurring polypeptide, wherein “biological” activity refers to a biological function (either inhibitory or stimulatory) caused by a native or naturally-occurring other than the ability to induce the production of an antibody against an antigenic epitope possessed by a native or naturally-occurring and an “immunological” activity refers to the ability to induce the production of an antibody against an antigenic epitope possessed by a native or naturally-occurring polypeptide.

6.14 Proteins and Polypeptides

A protein includes an isolated protein having a particular amino acid. The invention also includes a mutant or variant protein any of whose residues may be changed from the corresponding residue of the reference, or given, sequence while still encoding a protein that maintains its protein-like activities and physiological functions, or a functional fragment thereof. For example, the invention includes the polypeptides encoded by the variant nucleic acids described above. In the mutant or variant protein, up to 20% or more of the residues may be so changed.

In general, a protein-like variant that preserves protein-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a non-essential or conservative substitution as defined above. Furthermore, without limiting the scope of the invention, positions in a polypeptide may be substituted such that a mutant or variant protein may include one or more substitutions.

The invention also includes isolated proteins, and biologically active portions thereof, or derivatives, fragments, analogs or homologs thereof. Also provided are polypeptide fragments suitable for use as immunogens to raise anti-protein antibodies. A fragment of a protein or polypeptide, such as a peptide or oligopeptide, may be 5 amino acid residues or more in length, or 6 or more, 7 or more, 8 or more, 9 or more, 10 or more, 15 or more, 20 or more, 25 or more, 30 or more, 50 or more, 10 or more residues in length, up to a length that is one residue shorter than the full length sequence. In one embodiment, native proteins can be isolated from cells or tissue sources by an appropriate purification scheme using standard protein purification techniques. In another embodiment, proteins are produced by recombinant DNA techniques. Alternative to recombinant expression, a protein or polypeptide can be synthesized chemically using standard peptide synthesis techniques. Purification of proteins and polypeptides is described, for example, in texts such as “Protein Purification, 3^{rd} Ed.”, R. K. Scopes, Springer-Verlag, New York, 1994; “Protein Methods, 2^{nd} Ed.,” D. M. Bollag, M. D. Rozycki, and S. J. Edelsterin, Wiley-Liss, New York, 1996; and “Guide to Protein Purification”, M. Deutscher, Academic Press, New York, 2001.

Biologically active portions of a protein include peptides comprising amino acid sequences sufficiently similar to or derived from the amino acid sequence of a given protein that include fewer amino acids than the full length proteins, and exhibit at least one activity of a protein. Typically, biologically active portions comprise a domain or motif with at least one activity of the protein. A biologically active portion of a protein can be a polypeptide which is, for example, 10, 25, 50, 100 or more amino acids in length.

A biologically active portion of a protein of the present invention may contain at least one of the above-identified domains conserved among the family of proteins. Moreover, other biologically active portions, in which other regions of the protein are deleted, can be prepared by recombinant techniques and evaluated for one or more of the functional activities of a native protein.

In one embodiment, the protein has a given amino acid sequence. In another embodiment, the protein is substantially similar to the given sequence and retains the functional activity of the protein having the given sequence, yet differs in amino acid sequence due to natural allelic variation or mutagenesis, as described in detail below. In another embodiment, the protein is a protein that comprises an amino acid sequence at least about 45% similar, and more preferably about 55% or more, 65% or more, 70% or more, 75% or more, 80% or more, 85% or more, 90% or more, 95% or more, 98% or more, or even 99% or more similar to the disclosed amino acid sequence and retains the functional activity of the proteins of the corresponding polypeptide having the disclosed sequence. Non-limiting examples of particular amino acid residues that may changed in a variant polypeptide molecule are identified as the result of an alignment of a given polypeptide with a homologous or paralogous polypeptide.

6.15 Chimeric and Fusion Proteins

The invention also provides protein chimeric or fusion proteins. As used herein, a protein “chimeric protein” or “fusion protein” includes a polypeptide operatively linked to a non-polypeptide. A “polypeptide” refers to a polypeptide having an amino acid sequence corresponding to the protein, whereas a “non-polypeptide” refers to a polypeptide having an amino acid sequence corresponding to a protein that is not substantially similar to the protein, e.g., a protein that is different from the protein and that is derived from the same or a different organism. Within a fusion protein containing a protein the polypeptide can correspond to all or a portion of a protein. In one embodiment, a protein fusion protein comprises a full length protein or at least one biologically active fragment of a protein. In another embodiment, a protein fusion protein comprises at least two fragments of a protein each of which retains its biological activity. Within the fusion protein, the term “operatively linked” is intended to indicate that the polypeptide and the non-polypeptide are fused in-frame to each other. The non-polypeptide can be fused to the N-terminus or C-terminus of the polypeptide.

In another embodiment, the fusion protein is a GST-protein fusion protein in which the protein sequences are fused to the C-terminus of the GST (i.e., glutathione S-transferase) sequences. Such fusion proteins can facilitate the purification of recombinant protein. Additional fusion embodiments include FLAG-tagged fusions and fluorescent protein fusions, useful for purification and detection of the fusion construct.

In yet another embodiment, the fusion protein is a protein containing a heterologous signal sequence at its N-terminus. For example, the native protein signal sequence can be removed and replaced with a signal sequence from another protein. In certain host cells (e.g., mammalian host cells), expression and/or secretion of the protein can be increased through use of a heterologous signal sequence.

In another embodiment, the fusion protein is a protein-immunoglobulin fusion protein in which the protein sequences comprising one or more domains are fused to sequences derived from a member of the immunoglobulin protein family. The protein-immunoglobulin fusion proteins of the invention can be incorporated into pharmaceutical compositions and administered to a subject to inhibit an interaction between a protein ligand and a protein on the surface of a cell, to thereby suppress protein-mediated signal transduction in vivo.

A protein chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and re-amplified to generate a chimeric gene sequence (see, for example, Brent et al., Current Protocols in Molecular Biology, Wiley Interscience Publishers, (2003)). Moreover, many expression vectors are commercially available that already encode a fusion moiety (e.g., a GST polypeptide). A protein-encoding nucleic acid can be cloned into such an expression vector such that the fusion moiety is linked in-frame to the protein.

A “specific binding agent” of a polypeptide or a oligopeptide is any substance that specifically binds the polypeptide or oligopeptide, but binds weakly or not at all to other polypeptides and oligopeptides. Non-limiting examples of specific binding agents include antibodies, specific receptors for polypeptides, binding domains of such antibodies and receptors, aptamers, imprinted polymers, and so forth.

6.16 Detection and Labeling

A polynucleotide or a polypeptide may be detected in many ways. Detecting may include any one or more processes that result in the ability to observe the presence and or the amount of a polynucleotide or a polypeptide. In one embodiment a sample nucleic acid containing a polynucleotide may be detected prior to expansion. In an alternative embodiment a polynucleotide in a sample may be expanded to provide an expanded polynucleotide, and the expanded polynucleotide is detected or quantitated. Physical, chemical or biological methods may be used to detect and quantitate a polynucleotide. Physical methods include, by way of non-limiting example, optical visualization including various microscopic techniques such as fluorescence microscopy, confocal microscopy, microscopic visualization of in situ hybridization, surface plasmon resonance (SPR) detection such as binding a probe to a surface and using SPR to detect binding of a polynucleotide or a polypeptide to the immobilized probe, or having a probe in a chromatographic medium and detecting binding of a polynucleotide in the chromatographic medium. Physical methods further include a gel electrophoresis or capillary electrophoresis format in which polynucleotides or polypeptides are resolved from other polynucleotides or polypeptides, and the resolved polynucleotides or polypeptides are detected. Physical methods additionally include broadly any spectroscopic method of detecting or quantitating a substance. Chemical methods include hybridization methods generally in which a polynucleotide hybridizes to a probe. Biological methods include causing a polynucleotide or a polypeptide to exert a biological effect on a cell and detecting the effect. The present invention discloses examples of biological effects which may be used as a biological assay. In many embodiments, the polynucleotides may be labeled as described below to assist in detection and quantitation. For example, a sample nucleic acid may be labeled by chemical or enzymatic addition of a labeled moiety such as a labeled nucleotide or a labeled oligonucleotide linker. Many equivalent methods of detecting a polynucleotide or a polypeptide are known to workers of skill in fields related to the field of the invention, and are contemplated to be within the scope of the invention.

A nucleic acid of the invention can be expanded using cDNA, mRNA or alternatively, genomic DNA, as a template together with appropriate oligonucleotide primers according to any of a wide range of PCR amplification techniques. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

Polynucleotides, including expanded polynucleotides, may be detected and/or quantitated directly. For example, a polynucleotide may be subjected to electrophoresis in a gel that resolves by size, and stained with a dye that reveals its presence and amount. Alternatively a polynucleotide may be detected upon exposure to a probe nucleic acid under hybridizing conditions (see below) and binding by hybridization is detected and/or quantitated. Detection is accomplished in any way that permits determining that a polynucleotide has bound to the probe. This can be achieved by detecting the change in a physical property of the probe brought about by hybridizing a fragment. A nonlimiting example of such a physical detection method is SPR.

An alternative way of accomplishing detection is to use a labeled form of a polynucleotide or a polypeptide, and to detect the bound label. The polynucleotide may be labeled as an additional feature in the process of expanding the nucleic acid, or by other methods. A label may be incorporated into the fragments by use of modified nucleotides included in the compositions used to expand the fragment populations. A label may be a radioisotopic label, such as ¹²⁵I, ³⁵S, ³²P, ¹⁴C, or ³H, for example, that is detectable by its radioactivity. Alternatively, a label may be selected such that it can be detected using a spectroscopic method, for example. In one instance, a label may be a chromophore, absorbing incident light. A preferred label is one detectable by luminescence. Luminescence includes fluorescence, phosphorescence, and chemiluminescence. Thus a label that fluoresces, or that phosphoresces, or that induces a chemiluminscent reaction, may be employed. Examples of suitable fluorescent labels, or fluorochromes, include a ¹⁵² Eu label, a fluorescein label, a rhodamine label, a phycoerythrin label, a phycocyanin label, Cy-3, Cy-5, an allophycocyanin label, an o-phthalaldehyde label, and a fluorescamine label. Luminescent labels afford detection with high sensitivity.

A label may be a magnetic resonance label, such as a stable free radical label detectable by electron paramagnetic resonance, or a nuclear label, detectable by nuclear magnetic resonance. A label may still further be a ligand in a specific ligand-receptor pair; the presence of the ligand is then detected by the secondary binding of the specific receptor, which commonly is itself labeled for detection. Non-limiting examples of such ligand-receptor pairs include biotin and streptavidin or avidin, a hapten such as digoxigenin or antigen and its specific antibody, and so forth. A label still further may be a fusion sequence appended to a polynucleotide or a polypeptide. Such fusions permit isolation and/or detection and quantitation of the polynucleotide or a polypeptide. By way of non-limiting example, a fusion sequence may be a FLAG sequence, a polyhistidine sequence, a fluorescent protein sequence such as a green fluorescent protein, a yellow fluorescent protein, an alkaline phosphatase, a glutathione transferase, and the like. Labeling can be accomplished in a wide variety of ways known to workers of skill in fields related to the present disclosure. Any equivalent label that permits detecting and/or quantitation of a polynucleotide or a polypeptide is understood to fall within the scope of the invention.

Detecting, quantitating, including labeling, methods are known generally to those of skill in fields related to the present invention, including, by way of non-limiting example, workers of skill in spectroscopy, nucleic acid chemistry, biochemistry, molecular biology and cell biology. Quantitating permits determining the quantity, mass, or concentration of a nucleic acid or polynucleotide, or fragment thereof, that has bound to the probe. Quantitation includes determining the amount of change in a physical, chemical, or biological property as described in this and preceding paragraphs. For example, the intensity of a signal originating from a label may be used to assess the quantity of the nucleic acid bound to the probe. Any equivalent process yielding a way of detecting the presence and/or the quantity, mass, or concentration of a polynucleotide or fragment thereof that hybridizes to a probe nucleic acid is envisioned to be within the scope of the present invention.

6.17 Recombinant Vectors and Host Cells

Another aspect of the invention pertains to vectors, preferably expression vectors, containing a nucleic acid encoding protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively linked to the nucleic acid sequence to be expressed. Within a recombinant expression vector, “operably linked” is intended to mean that the nucleotide sequence of interest is linked to a regulatory sequence(s) in a manner that allows for expression of the nucleotide sequence (e.g., in an in vitro transcription/translation system or in a host cell when the vector is introduced into the host cell). The term “regulatory sequence” is intended to include promoters, enhancers and other expression control elements (e.g., polyadenylation signals). Such regulatory sequences are described, for example, in Goeddel (1990) GENE EXPRESSION TECHNOLOGY: METHODS IN ENZYMOLOGY 185, Academic Press, San Diego, Calif. Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., proteins, mutant forms of the protein, fusion proteins, etc.).

The recombinant expression vectors of the invention can be designed for expression of the protein in prokaryotic or eukaryotic cells. For example, the protein can be expressed in bacterial cells such as E. coli, insect cells (using baculovirus expression vectors) yeast cells or mammalian cells or suitable host cells. (Goeddel (1990) GENE EXPRESSION TECHNOLOGY: METHODS IN ENZYMOLOGY 185, Academic Press, San Diego, Calif.). Alternatively, the recombinant expression vector can be transcribed and translated in vitro, for example using T7 promoter regulatory sequences and T7 polymerase.

Promoter regions can be selected from any desired gene using vectors that contain a reporter transcription unit lacking a promoter region, such as a chloramphenicol acetyl transferase (“CAT”), or the luciferase (LUC) transcription unit, downstream of restriction site or sites for introducing a candidate promoter fragment; i.e., a fragment that may contain a promoter. For example, introduction into the vector of a promoter-containing fragment at the restriction site upstream of the CAT or LUC gene engenders production of CAT or LUC activity, respectively, which can be detected by standard CAT or LUC assays. Vectors suitable to this end are well known and readily available. Two such vectors are pKK232-8 and pCM7. Thus, promoters for expression of polynucleotides of the present invention include not only well-known and readily available promoters, but also promoters that readily may be obtained by the foregoing technique, using a reporter gene.

Expression of proteins in prokaryotes is most often carried out in E. coli with vectors containing constitutive or inducible promoters directing the expression of either fusion or non-fusion proteins. Among known bacterial promoters suitable for expression of polynucleotides and polypeptides are the E. coli lacI and lacZ promoters, the T3 and T7 promoters, the T5 tac promoter, the lambda PR, PL promoters and the trp promoter. Fusion vectors add a number of amino acids to a protein encoded therein, usually to the amino terminus of the recombinant protein. Such fusion vectors typically serve three purposes: (1) to increase expression of recombinant protein; (2) to increase the solubility of the recombinant protein; and (3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson (1988) Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein. Suitable inducible non-fusion E. coli expression vectors include pTrc (Amrann et al., (1988) Gene 69:301-315) and pET 11d (Studier et al., 1990 GENE EXPRESSION TECHNOLOGY: METHODs IN Enzymology 185, Academic Press, San Diego, Calif. 60-89).

In another embodiment, the expression vector is a yeast expression vector. Examples of vectors for expression in yeast S. cerivisae include pYepSec1 (Baldari, et al., (1987) EMBO J 6:229-234), pMFa (Kuran and Herskowitz, (1982) Cell 30:933-943), pJRY88 (Schultz et al., (1987) Gene 54:113-123), pYES2 (Invitrogen Corporation, San Diego, Calif.), and picZ (InVitrogen Corp, San Diego, Calif.).

Alternatively, the protein can be expressed in insect cells using baculovirus expression vectors. Baculovirus vectors available for expression of proteins in cultured insect cells (e.g., SF9 cells) include the pAc series (Smith et al. (1983) Mol Cell Biol 3:2156-2165) and the pVL series (Lucklow and Summers (1989) Virology 170:31-39).

In yet another embodiment, a nucleic acid of the invention is expressed in mammalian cells using a mammalian expression vector. Examples of mammalian expression vectors include pCDM8 (Seed (1987) Nature 329:840) and pMT2PC (Kaufman et al., (1987) EMBO J 6:187-195). When used in mammalian cells, the expression vector's control functions are often provided by viral regulatory elements. For example, commonly used promoters are derived from polyoma, Adenovirus 2, cytomegalovirus and Simian Virus 40. Other eukaryotic promoters include the CMV immediate early promoter, the HSV thymidine kinase promoter, the early and late SV40 promoters, the promoters of retroviral LTRs, such as those of the Rous sarcoma virus (“RSV”), and metallothionein promoters, such as the mouse metallothionein-I promoter. Those of skill in the art would be aware of other suitable expression systems for prokaryotic and eukaryotic cells. (See, e.g., Sambrook et al., MOLECULAR CLONING: A LABORATORY MANUAL. 3rd ed., Cold Spring Harbor Laboratory, Cold Spring Harbor Laboratory Press, Cold Spring Harbor, N.Y., 2001).

In another embodiment, the recombinant mammalian expression vector is capable of directing expression of the nucleic acid preferentially in a particular cell type. Non-limiting examples of suitable tissue-specific promoters include the albumin promoter (liver-specific; Pinkert et al., 1987 Genes Dev 1:268-277), lymphoid-specific promoters (Calame and Eaton, (1988) Adv Immunol 43:235-275), in particular promoters of T cell receptors (Winoto and Baltimore, (1989) EMBO J 8:729-733) and immunoglobulins (Banerji et al., 1983 Cell 33:729-740; Queen and Baltimore, (1983) Cell 33:741-748), neuron-specific promoters (e.g., the neurofilament promoter; Byrne and Ruddle, (1989) Proc. Natl. Acad. Sci. USA 86:5473-5477), pancreas-specific promoters (Edlund et al., (1985) Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, (1990) Science 249:374-379) and the α-fetoprotein promoter (Campes and Tilghman, (1989) Genes Dev 3:537-546).

The invention further provides a recombinant expression vector comprising a DNA molecule of the invention cloned into the expression vector in an antisense orientation. That is, the DNA molecule is operatively linked to a regulatory sequence in a manner that allows for expression (by transcription of the DNA molecule) of an RNA molecule that is antisense to a mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. For a discussion of the regulation of gene expression using antisense genes see Weintraub et al., “Antisense RNA as a molecular tool for genetic analysis,” Reviews—Trends in Genetics, Vol. 1(1) 1986.

6.18 Host Cells

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, the protein can be expressed in bacterial cells such as E. Coli, insect cells, yeast or mammalian cells (such as Chinese hamster ovary cells (CHO) or COS cells). Other suitable host cells are known to those skilled in the art.

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERO, BHK, HeLa, COS, MDCK, 293, 3T3, W138 cells, HEK293 cells, embryonic stem cells, adult origin stem cells, hematopoietic stem cells, tumor cells, cells from various mammalian organs, and the like.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (2001), Brent et al. (2003), and other laboratory manuals.

For stable transfection of mammalian cells, in order to identify and select stable integrants, a gene that encodes a selectable marker (e.g., resistance to antibiotics) is generally introduced into the host cells along with the gene of interest. Various selectable markers include those that confer resistance to drugs, such as G418, hygromycin and methotrexate.

6.19 Cell Culture

A cell culture to express is propagated using standard culture conditions. Twenty-four hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit or by FuGENE6 or by electroporation, calcium phosphate particle incorporation, or ballistic particles and expression is monitored using standard techniques with positive and negative control. A positive control is cells that naturally express the disclosed polynucleotide while a negative control is cells that do not express the polynucleotide.

A host cell of the invention, such as a prokaryotic or eukaryotic host cell in culture, can be used to produce (i.e., express) the protein. Accordingly, the invention further provides methods for producing the protein using the host cells of the invention. In one embodiment, the method comprises culturing the host cell of invention (into which a recombinant expression vector encoding the protein has been introduced) in a suitable medium such that the protein is produced. In another embodiment, the method further comprises isolating the protein from the medium or the host cell.

6.20 Multiplexed Genetic Analysis

High throughput genetic analyses, or genomic analyses, such as those contemplated in the present disclosure benefit from the ability to multiplex parallel assays in a single operation. This is accomplished by use of articles that include multiplexed arrays of genetic probes affixed to a single substrate, or articles that include assemblies of a plurality of identifiable objects, such as beads or particles, each of which includes a genetic probe affixed to it. Non-limiting examples, descriptions of the preparation, and use of arrays and beads include: U.S. Pat. No. 5,654,413; U.S. Pat. No. 5,429,807; U.S. Pat. No. 5,599,695; U.S. Pat. No. 6,309,823; U.S. Pat. No. 6,440,667; U.S. Pat. No. 6,355,432; U.S. Pat. No. 6,197,506; U.S. Pat. No. 6,309,822; U.S. Pat. No. 6,383,754.

The present disclosure provides methods that are advantageous in characterizing the functional genomics of alternative splice forms of multi-exon genes and gene products. The methods provide ways of helping identify genes of significance in cellular genomics and their prevalence in various tissues, organs and pathological states. Since there are approximately 30,000 mammalian genes and an average of 8 exons per gene, the present methods have the potential of focusing attention on those genes and splice variants important in functional analyses. The present inventors utilize a combined computational and experimental approach for EST data analyses. Particular embodiments of identifying exon junctions in selected genes, which are non-limiting with respect to the scope of the invention, are provided in Section 7.6-7.11.

6.21 Functional Genetic Analyses for Identifying Genes

The present invention provides methods for conducting a comprehensive genomic analysis of genetic factors underlying development of cell characteristics under altered conditions or as a result of differentiation. These methods provide a convenient, multiplexed analysis of genetic elements contributing to the changes in cell type. Additionally, the methods include analysis of the contributions of alternative splicing events contributing to these events.

In important embodiments of these methods cDNA is over-expressed in a subject cell. For example in identifying genes that are important in differentiation, a cDNA library is introduced into the cells using an episomal vector. To identify genes contributing to a new cell type, the transfected cells are exposed to altered or differentiation conditions. A fully saturated genetic analysis, i.e., a measurement of changes in relative representation of each transfected gene, is carried out using microarrays of oligonucleotide probes. The vector DNA is extracted from the transfected cells, before and after the analysis. Then, if necessary, the cDNA inserts are amplified by a method such as PCR. The DNA samples “before” and “after” the altered conditions were applied are labeled. Next, the labeled populations of DNA are hybridized to microarrays and changes in the tested cDNA population for each gene are recorded. It is estimated that two-fold differences and greater enrichment for each gene represented in the tested cDNA and present on the microarray can be determined. This will provide saturation analysis and detect all genes with strong and weak contributions to the studied cell type in a single experiment.

In many embodiments, the over-expression analyses described above employ oligonucleotide microarrays that are routinely used for genome-wide analysis of gene expression. Since the probes on such previously-devised arrays are designed toward the 3-prime end of the coding sequence, these arrays will not be able to distinguish splice variants originating from the same gene. Therefore, the present invention includes use of exon/exon-junction arrays. These arrays are constructed to sample representative sequences from each gene, including exon-exon junctions, and, thus, are able to provide a higher resolution for the genetic analyses.

Exon-exon junction arrays also become necessary for inhibition or loss of function analyses that utilize over-expression of cDNA fragment libraries, instead of libraries of full length cDNAs. The short cDNA fragments often encode dominant negative polypeptides that inhibit activity of the full-length proteins (holoproteins). (Roninson and Gudkov, 2003 Methods Mol. Biol. 222:413-436). In general, such cDNA fragment libraries include exon-exon junction sequences. The genetic analyses can be also applied to resolution of genetic analyses utilizing libraries of antisense nucleic acids, or of RNAi constructs. In such a case, the probe sequences on the exon microarray must be complementary to the antisense or RNAi sequences.

### 7.0 EXAMPLES

The following examples are provided for purpose of illustrating various embodiments of the invention and are not meant to limit the present invention.

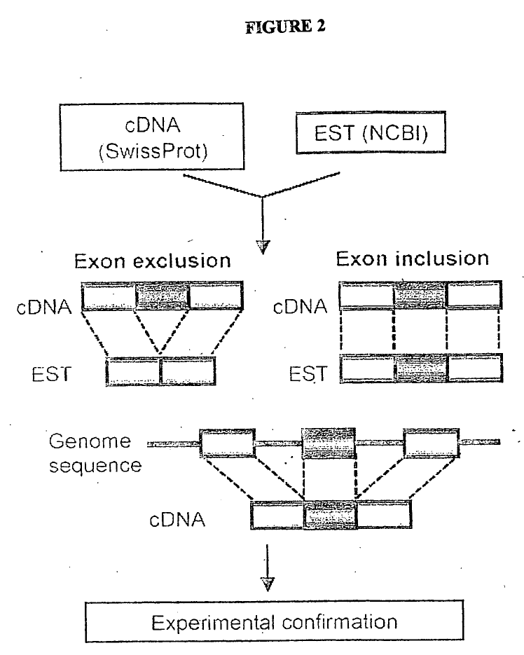

7.1 Computational Analysis of Alternative Splicing. The computational strategy for genome-wide analysis of alternative splicing is outlined in FIG. 2. Human cDNA and EST sequences were obtained from the SwissProt and NCBI EST databases. (Bairoch and Apweiler, (2000)Nucleic Acids Res. 28:45-48; Boguski et al., (1993) Nat. Gene 4:332-333). A reference set of 8,100 full-length cDNAs representing known human genes was derived from SwissProt, a manually annotated database (release 41). For each gene, the protein coding region contained within the longest available cDNA was selected. In order to avoid redundancy, pairs of genes that showed greater than 95% identity over at least 100 continuous nucleotides were removed from the reference set.

The BLAST program was used to align the ESTs with the reference cDNA set. Potential sites of alternative splicing were identified as gaps in the alignments. (Altschul et al., (1990) J. Mol. Biol. 215:403-410). For these studies, only inclusion or deletion of entire exons within protein coding regions was examined. A threshold of 95% identity over 100 nucleotides was used to define sequence identity. Alignment gaps were further selected by mapping to exon/intron boundaries in the human genome sequence (NCBI, build 32) using Spidey, a sensitive RNA-to-genome alignment program. (Wheelan et al., (2001) Genome Res. 11:1952-1957). FIG. 2 schematically illustrates origins of exon exclusion, leading to an alternative splicing event, as well as exon inclusion providing all exons in the final mRNA structure.

Using the above approach, 2,471 potential alternative splicing events were identified in 1,537 human genes represented in the reference set. In cases where a single gene included more than one site of alternative splicing, each site was analyzed separately. For each alternative splicing site the number of instances was calculated for which the given exon was included or excluded in the EST set. The exon exclusion fraction is defined as the number of ESTs with an excluded exon divided by the total number of ESTs spanning a site of alternative splicing.

7.2 RT-PCR Confirmation of Alternative Splicing. Selected instances of predicted alternative splicing were confirmed experimentally. Gene structures and orthologous relationships were obtained from the Ensemb1 database. (Hubbard et al., (2002) Nucleic Acids Res. 30:3841). Panels of total RNA from human adult tissues were purchased from BD Biosciences. RNA from cultured cells and some mouse tissues were obtained using the Trizol procedure (Invitrogen).

Random hexamer primers and MMLV reverse transcriptase (Invitrogen) were used for cDNA synthesis. Specific primers flanking predicted sites of alternative splicing were used for RT-PCR amplifications. TaqGold polymerase was used (Applied Biosystems) with the following PCR conditions: denaturation (95° C., 15 seconds), annealing (54° C., 30 seconds), elongation (72° C., 60 seconds).

A total of 25-29 cycles were performed. PCR products were separated on 1% agarose gel. Amplified GAPDH was used as loading control. The band intensities were quantified using the GelDoc imaging system and QuantityOne software (BioRad). Amplified products were cloned using the TOPO cloning kit (Invitrogen) and sequenced to confirm alternative splicing.

7.3 Cell Culture. Mouse CCE embryonic stem cells were cultured under standard conditions including 15% FBS and presence of Leukemia-Inhibitory Factor (LIF). Differentiation was induced upon removal of LIF.

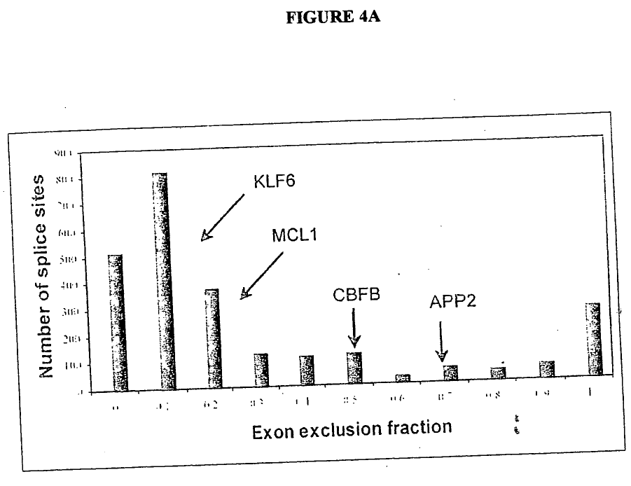

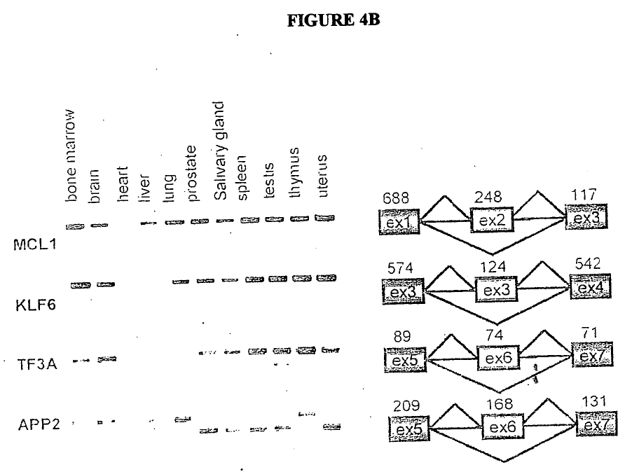

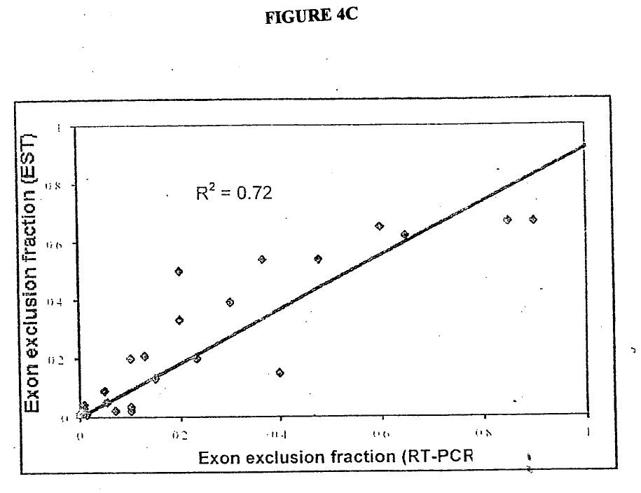

7.4 Design of Exon Microarrays. The microarray probes represented around 2,000 genes from the Ensembl database. (Hubbard et al., (2002) Nucleic Acids Res. 30:38-41). Target genes using keywords “transcription factor”, “cell cycle” and “apoptosis” were used to search Gene Ontology (GO) definitions. To avoid redundancy, exon sequences that show more than 85% similarity to other exons were filtered out. For each exon, two types of 60-mer probes (see FIG. 4A) were designed. The exon probe was complementary to the exon sequence and designed to detect exon exclusion as a decrease in the hybridization signal. The exon probe sequences were selected with the Agilent (Agilent Technologies, Palo Alto, Calif.) computational algorithm ensuring optimal hybridization parameters. The junction probes were complementary to 3′ and 5′ parts of flanking exons and designed to detect exon exclusion as an increase in the hybridization signal. For each exon, the junction probe was constructed by connecting 30-mer fragments of the 3′ and 5′ parts of the flanking exons. The probes were synthesized on glass microarrays by Agilent. The microarrays include positive and negative control probes.

7.5 cDNA synthesis. labeling and microarray hybridization. Total RNA was isolated from mouse tissues and cell cultures using the Trizol procedure (Invitrogen). mRNA was

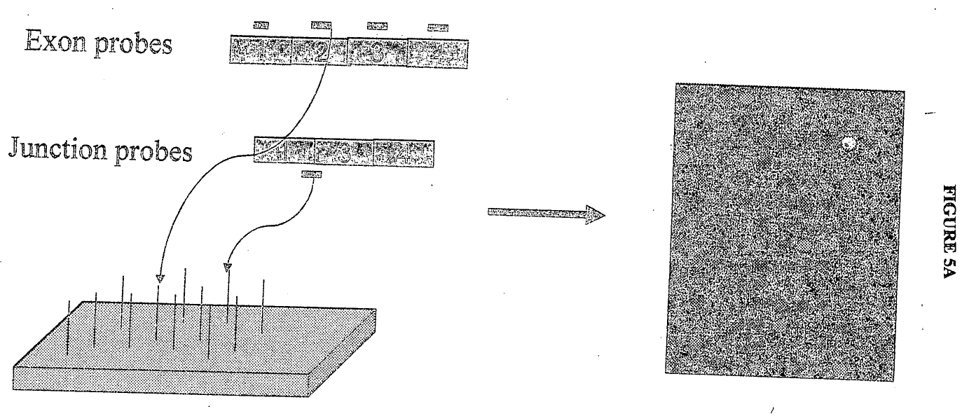

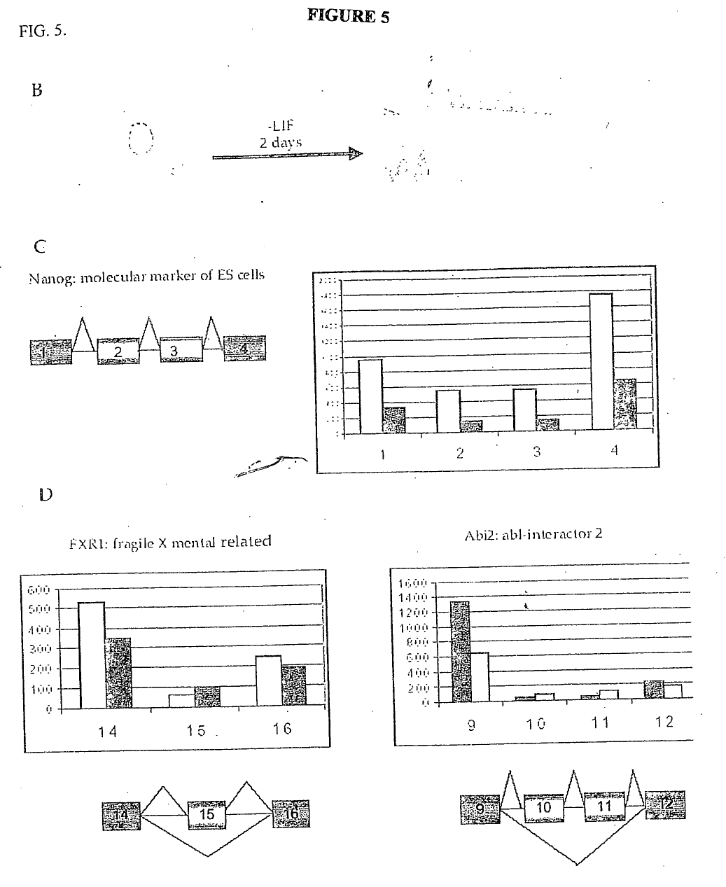

The following computational algorithm was applied to elucidate patterns of alternative splicing from the exon probe signal intensities (negative detection). Exon probes that satisfy the following conditions were searched for each gene:
I_{g}(non-diff)/I_{g}(diff)>2 and
Iₑ(non-diff)/Iₑ(diff)<1
where Iₑ was the signal from a particular exon and I_{g} was the average of the exon intensities across the gene. A situation in which one exon probe showed changes opposite to others indicated the occurrence of alternative splicing. RT-PCR was used to experimentally confirm predictions from the microarray data analyses. (see FIG. 5). Approximately 100 candidate splicing variations were identified using the current invention.

The previously described ES cell markers, Nanog and October-4, produced no splice variations as confirmed by RT-PCR. Therefore, a low hybridization signal was expected for their junction probes. Surprisingly, their junction probes showed hybridization signals as high as their exon probes (data not shown). Similar “false-positive” results were observed for junction probes from other genes. Therefore, the junction probes were not useful at the employed conditions. A certain level of noise for these probes was predetermined by their 50% identity to the 5′ and 3′ flanking exons. Currently, the hybridization conditions are being optimized in order to reduce the noise level. In addition, shortening of the junction probes may achieve an optimal signal-to-noise ratio. (Johnson et al., (2003) Science 302:2141-2144).

In contrast, examples of alternative splicing using exon junction microarrays, arising upon differentiation, were also demonstrated. (see FIG. 5, Panel D). It was shown that the computational algorithm (see Materials and Methods) had detected signal variations across the exon probes of the two genes, FXR1 and Abi2, which did not show proportional intensities across the exons examined. In addition, exon 15 of FXR1, and exons 10 and 11 of Abi2, exhibited very low intensities. This was interpreted, as arising from exon exclusion. (see lower line segments in the exon of FIG. 5). Alternative splicing were confirmed with RT-PCR.

7.9 Functional genomic analyses by saturation methods. A cDNA library was introduced into mouse ES cells using an episomal vector. To identify genes responsible for self-renewal, the transfected cells were exposed to differentiation conditions. Microarrays were utilized to measure

7.8 Detection of alternative splicing with exon microarrays. Alternative splicing was measured in mouse because both in vivo and in vitro experiments are feasible in this organism. Since high frequencies of alternative splicing are observed for regulatory genes, the probes were selected for 2,000 genes comprising more than one exon and defined as transcription, cell cycle or apoptosis regulators according to the Gene Ontology definitions.

Two types of microarray probes were designed (see FIG. 5, Panel A). Probes of the first type, “exon” probes (solid bars), were complementary to exons and, thus, are intended to account for exon loss as a result of alternative splicing. These probes were postulated to provide a negative detection of alternative splicing by measuring signal reduction for any excised exon. The probes of the second type, “junction” probes (diagonally shaded bar), were complementary to putative exon junctions that would form due to exclusion of a single exon and were intended to provide a positive detection for a splicing event. A mammalian gene comprises approximately 7-8 exons. Therefore, close to 44,000 probes for 2,000 genes were placed on two microarrays. The probes were oligonucleotides of 60 bases.

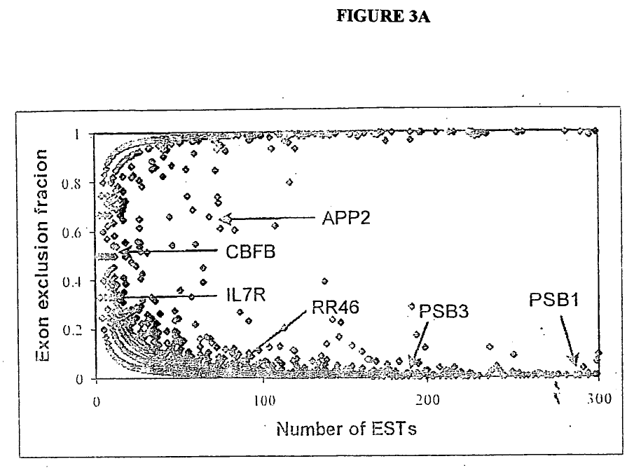

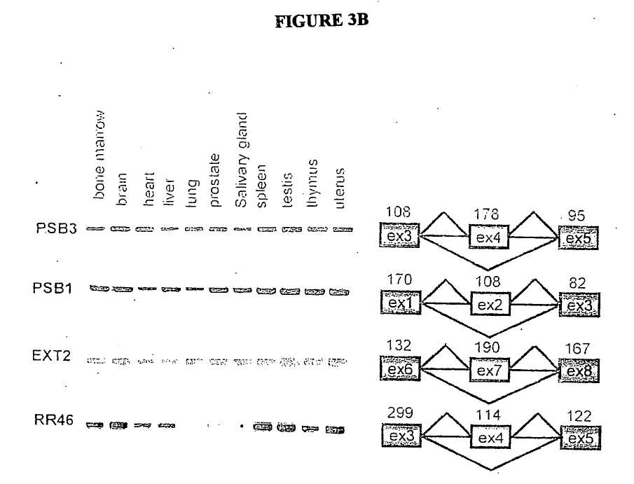

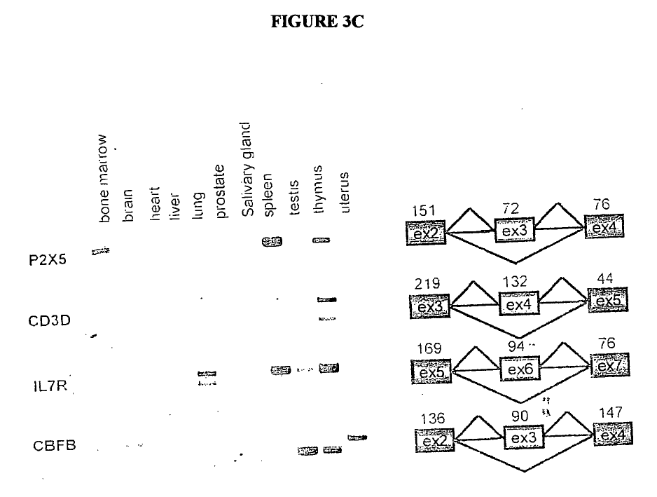

Alternative splicing was identified in embryonic stem (ES) cells, under non-differentiating and differentiating conditions. (see FIG. 5, Panel A). Differentiation of embryonic stem (ES) cells was performed using the reagents and hybridization conditions provided by Agilent. (see FIG. 5, Panel B). The differentiation was accompanied by a change in the characteristics of the cell. RNA was extracted from both non-differentiated and differentiated cells and applied to the microarray. The microarray data was analyzed for expression of known molecular markers of ES cells such as the gene encoding the transcriptional regulator Nanog. (see FIG. 5, Panel C). In Panel C, the left-most bar for each of the four exons was from the undifferentiated cells and the right-most bar was from the differentiated cells. As expected, these markers showed higher expression in undifferentiated ES cells. The changes in the signal intensity between the non-differentiated and differentiated states were similar for the four probes. As shown by the similar ratios for the two bars for each exon, Nanog was not alternatively spliced. This was demonstrated with the large signal variations between different exon probes from the same gene. (see FIG. 5). These results cannot be explained by unequal amplification of 5′ and 3′ transcript ends. A more plausible explanation is that these variations originate from the physiochemical differences between the probes as employed on the microarrays and the necessity of using one set of hybridization conditions for the entire array. experimentally, some of these genes did not show detectable splicing variants while others showed a very low frequency of alternative splicing. In contrast, a higher frequency of alternative splicing was observed and confirmed for genes with tissue-restricted expression (see FIG. 3, Panel C). Tissue specific genes revealed more equal frequencies of exon exclusion and inclusion. Selected examples include the ion channel P2X5, the hematopoietic transmembrane receptors IL7R and CD3D, and the transcription factor subunit CBFB. For these genes, the ratios of exon exclusion to inclusion were closer to 0.5. In general, these genes represent regulatory functional categories such as transcription factors, transmembrane receptors, kinases and others. However, there are exceptions. As shown in FIG. 4, Panel B, there are clearly examples of non-tissue specific genes with ratios close to 0.5.

The inclusion or exclusion frequencies of individual exons were distributed in a highly non-random fashion. (see FIG. 4, Panel A). The fraction of long isoforms (exon inclusion) was greater than 0.8 in nearly 60% of the analyzed alternative splice sites. At most sites of alternative splicing, exon exclusion was a rare event. Interestingly, very few sites showed an equal representation of both alternatively spliced isoforms. These observations were confirmed for 25 genes representing different predicted ratios of exon inclusion to exclusion. As expected, the majority of the experimentally tested genes showed predominant formation of the long isoforms, while exon exclusion was rare and tissue-specific.

The apoptosis gene MCL1 and the transcription factors TF3A and KLF6 represent examples in most tissues of predominant exon inclusion. (see FIG. 4, Panel B). Weak bands representing exon exclusion events were also found. RT-PCR analysis of transcripts from multiple tissues revealed that exon exclusion in these genes was tissue specific. The gene encoding amyloid-like protein APP2 revealed approximately equal exon inclusion and exclusion frequencies. For most genes, exon inclusion (upper band) is prevalent, in comparison to exon exclusion (see FIG. 4, Panel B, upper band and lower band, respectively). In general, the computationally determined exon exclusion frequencies were in agreement with the experimental data (see FIG. 4, Panel C). The exon exclusion fractions, calculated with the EST data and determined experimentally with RT-PCR, were plotted versus each other. The regression line had a slope 0.92, r-squared 0.72. isolated using the Oligotex kit (Qiagen). mRNA quality was tested with the denaturing gel and Northern blot. cDNA was synthesized and converted to fluorescently labeled cRNA according to the Agilent protocol. The sample hybridization was performed according to the Agilent protocol. Hybridization intensities were measured with a GenePix® scanner (Axon Instruments, Union City, Calif.).

7.6 Analysis of Alternative SPlicing. Using EST sequence alignments, 2,471 alternative splice sites were computationally identified within coding regions of 8,100 human genes (see FIG. 2). A set of 30 genes was chosen for experimental confirmation. These genes encode proteins of various functions and were chosen to represent different levels of expression as represented by the corresponding ESTs (see FIG. 3, Panel A). Because ESTs originate from random sequencing efforts, the number of ESTs corresponding to a particular gene is representative of its expression level. Thus, ubiquitous and tissue-specific genes are represented by high and low numbers of ESTs, respectively. The RT-PCR data in Panel A support this reasoning. Inclusion or exclusion of one or more exons yields a long or short isoform, respectively, for each analyzed gene.

In order to test for correlations between the levels of transcription and alternative splicing, the exon exclusion fractions at each splice site were analyzed as a function of the number of ESTs covering this site. The calculations were performed across all tissues because tissue-specific EST libraries are not sufficiently comprehensive.

7.7 Experimental Correlation between Transcription and Splicing. RNA samples from 11 tissues were utilized. Computationally predicted splice variants were confirmed in 80% of the 30 genes by RT-PCR and sequencing. Representative examples of confirmed alternative splicing events are presented in FIGS. 3 and 4. As shown in FIG. 3, Panel A, ubiquitously expressed genes show a low frequency of alternative splicing; that is, most are represented by a single isoform. This observation was experimentally tested for 8 ubiquitously expressed genes with computationally detected splice variants (see FIG. 3, Panel B). The locations of the experimentally tested genes are shown in Panel A with arrows.

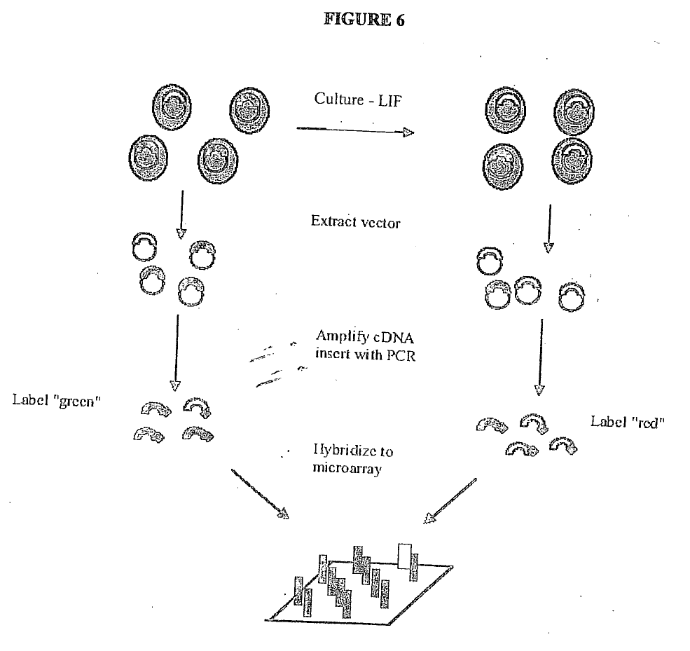

The set of genes includes those that encode the proteosome components PSB 1 and PSB3, the exosomal component RR46 and glucosyl transferase EXT2. According to computational results, the exon exclusion fractions were close to 0.01 for these genes. When tested changes in relative representation of each overexpressed gene. (see FIG. 6). To conduct the analysis, the ES cells, which were cultured in the presence of LIF, were transferred to medium free of LIF. The vector DNA from the transfected cells before and after the analysis was extracted. The cDNA inserts were amplified with PCR and the “before” and “after” samples are labeled with the fluorochromes Cy-5 and Cy-3, respectively. The labeled populations were hybridized to microarrays that included exon/exon-junction probes that represented the entire sequence of each gene and recorded changes in the tested cDNA population for each gene. It was expected that two-fold and greater enrichment for each gene could be determined in the tested cDNA and presented on the microarray. This achieved saturation genetic analysis and detected all genes with strong and weak contributions to the studied cell type in a single experiment.

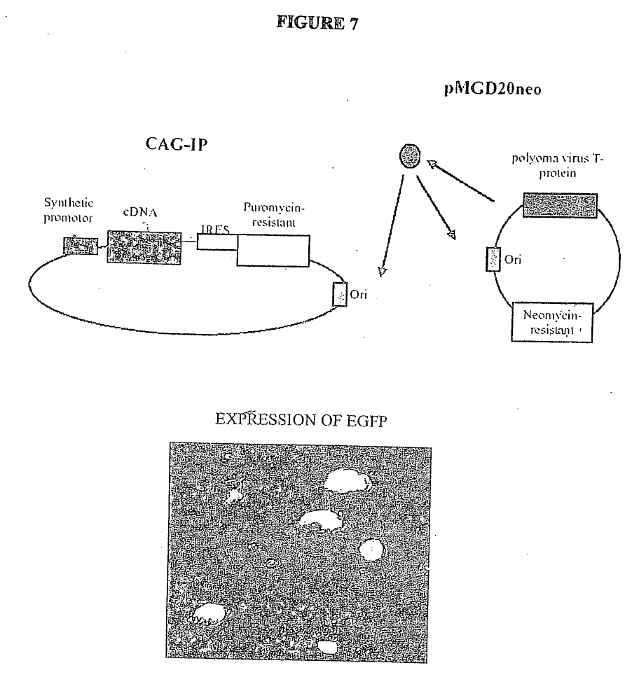

In a particular embodiment of this example, a cDNA library was constructed from mouse embryo at day 11 RNA. The library was introduced into an episomal expression vector, CAG-IP, which was able to propagate in a modified ES cell line, MG1.19 (see FIG. 7). Sequencing randomly chosen clones determined that approximately 50% of the cDNA inserts were full-length. The library was transfected into the MG1.19 cells by electroporation. Under non-differentiating conditions, in the presence of serum and Leukemia Inhibitory factor (LIF), these cells maintained a characteristic morphological phenotype and proliferate with rapid kinetics (self-renew). FIG. 7 (lower panel) shows expression of EGFP in transfected MG1.19 cells.

The transfected cells were exposed to conditions that induced differentiation by the removal of LIF from the culture medium. After 12 days, vector DNA was extracted, labeled and hybridized to microarrays that included junction probes to identify splice variants and other genes responsible for stem cell self-renewal in the presence of LIF.

7.10 Loss of function genomic analyses by saturation methods. Microarrays containing junction probes provide high resolution probes useful for genetic analyses. These arrays also become necessary for “loss-of-function” analyses that utilize over-expression of cDNA fragment libraries. The short cDNA fragments often encode dominant negative proteins and inhibit activity of the full-length proteins. (Roninson and Gudkov, (2003) Methods Mol. Biol. 222:413-436). Experiments were carried out as described in Section 7.9 with the following modifications: i) the library used was a library of cDNA fragments and ii) the analysis identifies differences in genetic loss of function as the result of the change in culture conditions.

7.11 Inhibition of function genomic analyses by saturation methods. The genomic analyses described in Section 7.9 and 7.10 were applied to carry out genetic analyses utilizing a library of RNAi constructs instead of a cDNA library (see Section 7.9) or a library of cDNA fragments (see Section 7.10). In these instances, the probe sequences on the exon microarray must be complementary to the RNAi sequences.

All references cited herein are incorporated herein by references in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

### REFERENCES

1. Altschul, S. F., Gish, W., Miller, W., Myers, E. W. and Lipman, D. J. 1990. Basic local alignment search tool. J. Mol. Biol. 215:403-410.
2. Bairoch, A. and R. Apweiler. 2000. The SWISS-PROT protein sequence database and its supplement TrEMBL in 2000. Nucleic Acids Res. 28:45-48.
3. Black, D. L. 2000. Protein diversity from alternative splicing: a challenge for bioinformatics and post-genome biology. Cell 103:367-370.
4. Boguski, M. S., Lowe, T. M. and Tolstoshev, C. M. 1993. dbEST-databse for “expressed sequence tags”. Nat Gene 4:332-333.
5. Clark, T. A., Sugnet, C. W. and M. Ares, Jr. 2002. Genome-wide analysis of mRNA processing in yeast using splicing-specific microarrays. Science 296:907-910.
6. Graveley, B. R. 2001. Alternative splicing: increasing diversity in the proteomic world. Trends Genet. 17:100-107.
7. Hubbard, T., Barker, D., Birney, G., et al., 2002. The Ensemb1 genome database project. Nucleic Acids Res. 30:38-41.
8. Johnson, J. M., Castle, P., Garrett-Engele, Z., et al., 2003. Genome-wide survey of human alternative pre-mRNA splicing with exon junction microarrays. Science. 302:2141-2144.
9. Maniatis, T. and B. Tasic. 2002. Alternative pre-mRNA splicing and proteome expansion in metazoans. Nature 418:236-243.
10. Modrek, B. and C. Lee. 2002. A genomic view of alternative splicing. Nat. Genet. 30:13-19.
11. Modrek, B., Resch, C., Grasso, C. and Lee, C. 2001. Genome-wide detection of alternative splicing in expressed sequences of human genes. Nucleic Acids. Res. 29:2850-2859.
12. Roninson, I. B. and A. V. Gudkov. 2003. Genetics suppressor elements in the characterization and identification of tumor suppressor genes. Methods Mol. Biol. 222:413-436.
13. Sharp, P. A. 1994. Split genes and RNA splicing. Cell 77:805-815.
14. Wheelan, S. J., Church, D. M. and J. M. Ostell. 2001. Spidey: A Tool for mRNA-to-genomic alignments. Genome Res. 11:1952-1957.
15. Yeakley, J. M., Ran, J. B., Doucet, D., et al. 2002. Profiling alternative splicing on fiber-optic arrays. Nat. Biotechnol. 20:353-358.
16. Zavolan, M. E., van Nimwegen, E. and T. Gaasterland. 2002. Splice variation in mouse full-length cDNAs identified by mapping to the mouse genome. Genome Res. 12:1377-1385.

What is claimed is:

1. A method for identifying a polynucleotide sequence comprising an exon junction, comprising the steps of:
a) providing a sample comprising one or more polynucleotides;
b) probing the polynucleotide with probe polynucleotides wherein at least one probe polynucleotide comprises a junction resulting from exon exclusion; and
c) determining that a probe comprising a junction binds a polynucleotide present in the polynucleotides
thereby identifying the polynucleotide sequence.

2. A method for identifying a polynucleotide sequence comprising an exon junction present in a second sample cell that is substantially absent or present in a reduced amount in a first sample cell, comprising the steps of:
a) introducing a library of exogenous polynucleotide sequences into first sample cells and into second sample cells;
b) culturing the first sample cells and second sample cells;
c) isolating first polynucleotides from the first sample cells and second polynucleotides from the second sample cells;
d) probing the first polynucleotides and the second polynucleotides with probe polynucleotides wherein at least one probe polynucleotide comprises a junction resulting from exon exclusion; and
e) determining that a probe comprising an exon junction binds one or more second polynucleotides and binds no, or a substantially reduced amount of one or more first polynucleotides
thereby identifying the polynucleotide sequence.

3. The method according to claim 2, wherein the first sample cell and the second sample are from the sample species.

4. The method according to claim 2, wherein the first sample cell has a different phenotype from the second sample cell.

5. The method according to claim 2, wherein the probe polynucleotides further comprise at least one probe polynucleotide comprising a mono-exonic sequence.

6. The method according to claim 2, wherein a polynucleotide component of the library comprises a cDNA, a fragment of a cDNA, or an inhibitory polynucleotide.

7. A method for identifying a polynucleotide sequence comprising an exon junction, comprising the steps of:
a) introducing a library of exogenous polynucleotide sequences into a plurality of starting sample cells cultured under starting conditions;
b) applying altered culture conditions, effective to change the starting sample cells into altered sample cells, to at least a portion of the starting sample cells;
c) isolating starting polynucleotides from the starting sample cells and altered polynucleotides from the altered sample cells;
d) probing the starting polynucleotides and the altered polynucleotides with probe polynucleotides wherein at least one probe polynucleotide comprises a junction resulting from exon exclusion; and
e) determining that a probe comprising an exon junction binds one or more starting polynucleotides and binds no, or a substantially reduced amount of one or more of the altered polynucleotides, or that a probe comprising an exon junction binds one or more altered polynucleotides and binds no, or a substantially reduced amount of one or more of the starting polynucleotides;
thereby identifying the polynucleotide sequence.

8. The method according to claim 7, wherein the first sample cell and the second sample are from the sample species.

9. The method according to claim 7, wherein the first sample cell has a different phenotype from the second sample cell.

10. The method according to claim 7, wherein the probe polynucleotides further comprise at least one probe polynucleotide comprising a mono-exonic sequence.

11. The method according to claim 7, wherein a polynucleotide component of the library comprises a cDNA, a fragment of a cDNA, or an inhibitory polynucleotide.

12. The method according to claim 7, wherein the method identifies polynucleotides that stimulate cell growth or that inhibit cell growth upon comparing polynucelotides isolated from altered cell samples to polynucleotides isolated from starting cells.
